(19) United States
(12) Patent Application Publication
(10) Pub. No.: US 2026/0083387 A1
(43) Pub. Date: Mar. 26, 2026

(54) MOTOR INTENTION PREDICTION

(71) Applicant: Apple Inc., Cupertino, CA (US)

(72) Inventors: Stephen Meisenhelter, San Francisco, CA (US); Hyun Jong Won, Sunnyvale, CA (US); Brian Pasley, Berkeley, CA (US); Izzet B. Yildiz, Sunnyvale, CA (US); Nikola Vukovic, San Francisco, CA (US); Grant H. Mulliken, Los Gatos, CA (US); Eva Pilar Esteban Velasco, San Francisco, CA (US)

(21) Appl. No.: 19/339,195

(22) Filed: Sep. 24, 2025

Related U.S. Application Data
(60) Provisional application No. 63/699,756, filed on Sep. 26, 2024.

(51) Int. Cl. A61B 5/375; A61B 5/00; A61B 5/384

(52) U.S. Cl. CPC A61B 5/375; A61B 5/384; A61B 5/7264; A61B 5/7275; A61B 5/7282

(57) ABSTRACT

Various implementations disclosed herein include devices, systems, and methods that determine an interaction event during presentation of an interaction element. For example, an example process may include obtaining physiological data associated with neurological signals during presentation of content to a user at the device, the content including an interaction element (e.g., a selectable icon). The process may further include predicting that the user is thinking of performing a physical act with a portion of a body of the user based on the physiological data, the performance of the physical act being associated with a particular type of interaction and is detectable based on sensor data (e.g., a pinch-based selection). The process may further include determining user interaction feedback corresponding to an interaction event associated with the interaction element based on the predicting that the user is thinking of performing the physical act.

### CROSS-REFERENCE TO RELATED APPLICATIONS

This Application claims the benefit of U.S. Provisional Application Ser. No. 63/699,756 filed Sep. 26, 2024, which is incorporated herein in its entirety.

### TECHNICAL FIELD

The present disclosure generally relates to presenting content via electronic devices, and in particular, to systems, methods, and devices that determine an interaction event by predicting motor intentions based on the presentation of electronic content and physiological data.

### BACKGROUND

Determining a user's intent while viewing content on an electronic device can facilitate a more meaningful experience. For example, a user interface element (e.g., a selectable icon or button) may be automatically selected based on determining the user's intent to make such a selection and without the user necessarily having to perform a gesture, mouse click, or other input-device-based action to initiate the selection. Improved techniques for assessing the intent of users viewing and interacting with content may enhance the users'enjoyment, comprehension, and learning of the content. Content creators and systems may be able to provide better and more tailored user experiences based determining user intent to interact with user interface elements.

### SUMMARY

Various implementations disclosed herein include devices, systems, and methods that assess neurological signals to predict a user interaction event (e.g., predicting a motor intention—such as pinch for a click) on a particular user interface element (e.g., predicting when a user is focused on a particular portion of the content). For example, a method may identify that, during a particular segment of an experience, the user's neurological signals (e.g., electroencephalogram (EEG) data) correspond to a user focusing on and selecting a particular icon or user interface element (e.g., merely imagining motion or intending to move may generate similar signals in the motor cortex). In other words, detecting a click intention only using electrical activity in the brain, and not requiring an input device or hand interactions (e.g., a pinch-based movement). For EEG, this may be based on monitoring signals from the motor cortex and other regions. For example, a user interaction event (e.g., click) may be based on (EEG) signals while wearing a head-mounted device (HMD).

In some implementations, other physiological data (e.g., gaze characteristic(s), head/body movement data, and the like) and illumination characteristics of an interaction element may also be used to predict an interaction event. For example, a user may direct their attention to a bright feature in an icon or other user interface element in order to initiate a “click” or other interaction. Physiological data may be used to determine an interaction event. For example, some implementations may identify that the user's eye characteristics (e.g., blink rate, stable gaze direction, saccade amplitude/velocity, and/or pupil radius) relate to an interaction with a presentation of an interaction element (e.g., an icon) based on a user's focus upon different regions of the interaction element that have different illumination characteristics. For example, the illumination features may include relatively dark or bright regions. For example, a method may identify that, during a particular segment of the experience, the user's gaze characteristics (e.g., pupil dilation vs. constriction, stable gaze direction and/or velocity) corresponds to a user focusing on a particular icon or user interface element. Additionally, determining the user's eye characteristics may involve obtaining images of the eye or electrooculography (EOG) data, microsaccades, and/or head movements, from which pupil response/gaze direction/movement can be determined.

In some implementations, determining an interaction event may be based on a characteristic of an environment of the user (e.g., real-world physical environment, a virtual environment, or a combination of each). The device (e.g., a handheld, laptop, desktop, or head-mounted device (HMD)) provides an experience (e.g., a visual and/or auditory experience) of the real-world physical environment or an extended reality (XR) environment. The device obtains, with one or more sensors, physiological data (e.g., electroencephalography (EEG) amplitude, electromyography (EMG), pupil modulation, eye gaze saccades, head movements measured by an inertial measurement unit (IMU), etc.) associated with the user. Based on the obtained physiological data, the techniques described herein can determine an interaction event during the experience. Based on the physiological data and associated physiological response (e.g., a user focusing on a particular region of the content), the techniques can provide a response to the user based on the interaction event and adjust the content corresponding to the experience.

Physiological response data, such as EEG amplitude/frequency, pupil modulation, eye gaze saccades, etc., can depend on the individual, characteristics of the scene in front of him or her (e.g., video content), and attributes of the physical environment surrounding the user including the activity/movement of the user. Physiological response data can be obtained while using a device with eye tracking technology (and other physiologic sensors) while users perform tasks. In some implementations, physiological response data can be obtained using other sensors, such as EEG sensors, EMG sensors, EDA sensors, and the like, that may obtain physiological signals from the head, limbs, or other portions of a person. Observing repeated measures of physiological response data to an experience can give insights about the intent of the user.

Several different experiences can utilize the techniques described herein regarding assessing interaction events using a cognitive based user interface. For example, the method can be provided to support users who want to interact with user interface elements using neurological signals without using hands, voice, or overt eye movements like dwell time. Additionally, determining interaction events can be used as an accessibility feature, for example, that enables paralyzed users to interact by selecting computer graphic icons using neurological signals (e.g., think of making a pinch movement for a particular icon) and/or eye gaze data. Additionally, determining interaction events can be used in general applications (e.g., a user interface selection tool, a device wake-up signal, etc.), and might be combined with other eye or touch-based mechanisms, such as to improve signal-to-noise ratio (SNR), robustness, response time, and the like. The cognitive based user interface may utilize two learning systems: i) learn to more accurately detect the neural signal over time, and ii) train the user to produce clearer neural signals (e.g., enhance the accuracy and sensitivity of a neural pinch signal for each user).

Context may additionally be used to determine interaction events. For example, a scene analysis of an experience can determine a scene understanding of the visual and/or auditory attributes associated with content being presented to the user (e.g., what is being presented in video content) and/or attributes associated with the environment of the user (e.g., where is the user, what is the user doing, what objects are nearby). These attributes of both the presented content and environment of the user can improve the determination of the user's intent regarding an interaction event.

Some implementations focus on improving the accuracy for assessing interaction events based on a user's neurological signals, pupillary response, body/head movement data, and the like, by incorporating practice exercises. For example, a machine learning algorithm may be implemented to determine whether or not a user's focus/intent means that he or she is intending to select a particular icon (e.g., motor intention prediction).

Some implementations assess physiological data and other user information to help improve a user experience. In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user should have the ability to access and otherwise find out anything that the system has collected or determined about him or her.

In general, one innovative aspect of the subject matter described in this specification can be embodied in methods, at a device including a processor, that include the actions of obtaining physiological data associated with neurological signals during presentation of content to a user at the device, wherein the content includes an interaction element. The actions may further include predicting, based on the physiological data, that the user is thinking of performing a physical act with a portion of a body of the user, wherein performance of the physical act is associated with a particular type of interaction and wherein performance of the physical act is detectable based on sensor data. The actions may further include determining user interaction feedback corresponding to an interaction event associated with the interaction element based on the predicting that the user is thinking of performing the physical act.

These and other embodiments can each optionally include one or more of the following features.

In some aspects, predicting that the user is thinking of performing the physical act is based on recognizing that a pattern exhibited in the neurological signals of the physiological data is indicative of the user thinking of performing the physical act.

In some aspects, the actions may further include, and in response to determining, based on the sensor data, that the physical act associated with the particular type of interaction is performed, obtaining additional physiological data associated with neurological signals, and updating a prediction model associated with the user performing the physical act associated with the particular type of interaction based on the additional physiological data.

In some aspects, the actions may further include, and in response to determining, based on the sensor data, that the physical act associated with the particular type of interaction is not performed while the user is thinking of performing the physical act, obtaining additional physiological data associated with neurological signals, and updating a prediction model associated with the user performing the physical act associated with the particular type of interaction based on the additional physiological data.

In some aspects, predicting that the user is thinking of performing the physical act with the portion of the body of the user is based on identifying a neurological event with at least one of the neurological signals. In some aspects, identifying the neurological event with the at least one neurological signal includes determining whether one or more components of the at least one neurological signal includes a change in one or more attributes with respect to a threshold.

In some aspects, the physiological data associated with neurological signals includes electroencephalogram (EEG) data. In some aspects, the device further includes at least three neurological sensors configured to obtain the physiological data associated with neurological signals corresponding to motor cortex signals. In some aspects, each sensor of the at least three neurological sensors are positioned at different regions of the device. In some aspects, the different regions of the device that each of the at least three neurological sensors are positioned includes a support element of the device, on a user facing surface adjacent to a lens of the device, via an additional support element coupled to the device, or a combination thereof.

In some aspects, the user interaction feedback corresponding to an interaction event associated with the interaction element is classified using a machine learning technique. In some aspects, the physiological data includes pupillary data, and wherein predicting that the user is thinking of performing the physical act is further based on determining a pupillary response associated with the interaction element. In some aspects, the pupillary response is a direction of the pupillary response, a velocity of the pupillary response, or pupillary fixations.

In some aspects, predicting that the user is thinking of performing the physical act with the portion of the body of the user is based on a combination of neurological signals corresponding to a neurological response and pupillary data corresponding to a pupillary response.

In some aspects, the actions may further include, prior to presenting the user interaction feedback, filtering a prediction value indicative of a motor intention through a damped-spring control function to generate a smoothed feedback signal. In some aspects, the smoothed feedback signal is based on at least one of a visual, haptic, and an auditory output presented to the user.

In some aspects, the user interaction feedback includes rendering a virtual representation of a portion of the user, the representation being animated toward the physical act in proportion to a confidence that the user is thinking of performing the physical act. In some aspects, the virtual representation is displayed adjacent to an interaction element predicted to be an intended target. In some aspects, updating the prediction model includes detecting a change in the motor-cortex signal after presentation of the virtual representation and adjusting model parameters based on the detected change.

In some aspects, the actions may further include modifying content in response to determining user interaction feedback corresponding to an interaction event associated with the interaction element based on the predicting that the user is thinking of performing the physical act.

In some aspects, the actions may further include obtaining additional physiological data associated with body movements, and updating a prediction model associated with predicting that the user is thinking of performing the physical act associated with the particular type of interaction based on the additional physiological data.

In some aspects, the device is a head-mounted device (HMD). In some aspects, the presentation of content is an extended reality (XR) experience that is presented to the user.

In accordance with some implementations, a non-transitory computer readable storage medium has stored therein instructions that are computer-executable to perform or cause performance of any of the methods described herein. In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates an environment in which extended reality (XR) content is provided to one or more users wearing head mounted displays (HMDs) in accordance with some implementations.

FIGS. 2A and 2B illustrate detecting an interaction event while viewing content based on physiological data in accordance with some implementations.

FIGS. 3A and 3B illustrate detecting an interaction event with a cognitive based user interface based on neurological data in accordance with some implementations.

FIG. 4 illustrates neurological signals for a neurological sensor system during an experience in accordance with some implementations.

FIG. 5 illustrates specific neurological band signals while a user is thinking of performing a physical action during an experience in accordance with some implementations.

FIG. 6 illustrates damped spring feedback graphs based on neurological signals while a user is thinking of performing a physical action during an experience in accordance with some implementations.

FIG. 7 illustrates detecting an interaction event with a cognitive based user interface and providing a view of a virtual limb based on neurological data in accordance with some implementations.

FIG. 8 illustrates a system diagram for motor intention predictions corresponding to an interaction event based on physiological data in accordance with some implementations.

FIG. 9 is a flowchart representation of a method for determining user interaction feedback corresponding to an interaction event by predicting physical act intentions based on neurological signals in accordance with some implementations.

FIG. 10 illustrates device components of an exemplary device in accordance with some implementations.

FIG. 11 illustrates an example head-mounted device (HMD) in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects or variants do not include all of the specific details described herein. Moreover, well-known systems, methods, components, devices and circuits have not been described in exhaustive detail so as not to obscure more pertinent aspects of the example implementations described herein.

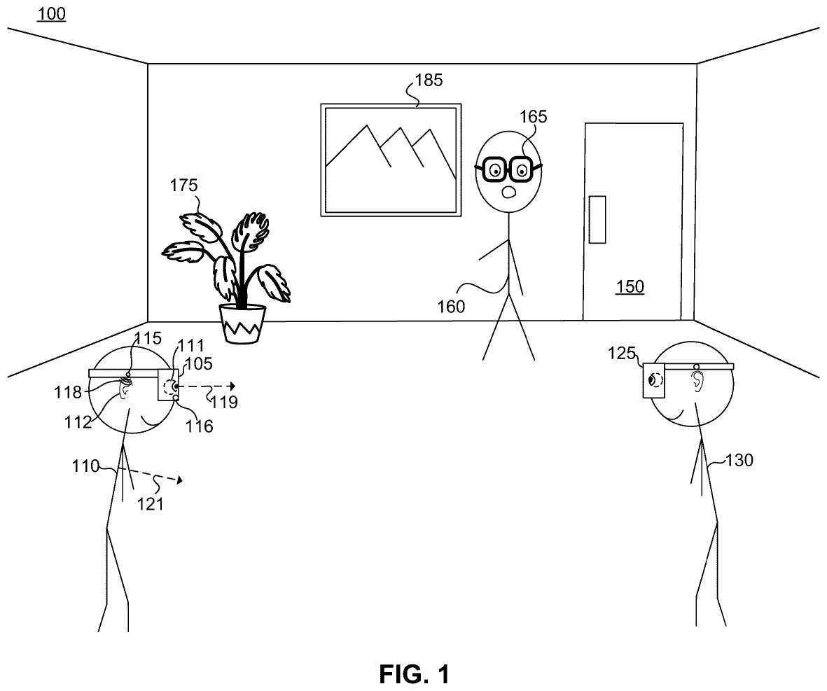

FIG. 1 illustrates a real-world physical environment 100 including a first user 110 wearing a first device 105, a second user 130 wearing a second device 125, a third user 160 wearing a third device 165, a wall-hung picture 185, a plant 175, and a door 150. In some implementations, one or more of the devices 105, 125, 165 is configured to provide content based on one or more sensors on the respective devices or to share information and/or sensor data with one another. In some implementations, one or more of the devices 105, 125, 165 provide content that provides augmentations in XR using sensor data. The sensor data may be used to understand that a user's state is associated with providing user assistance, e.g., a user's appearance or behavior or an understanding of the environment may be used to recognize a need or desire for assistance.

In the example of FIG. 1, the first device 105 includes one or more sensors 116 that capture light-intensity images, depth sensor images, audio data or other information about the user 110 and the physical environment 100. For example, the one or more sensors 116 may capture images of the user's forehead, eyebrows, eyes, eye lids, cheeks, nose, lips, chin, face, head, hands, wrists, arms, shoulders, torso, legs, or other body portion. Sensor data about a user's eye 111, as one example, may be indicative of various user characteristics, e.g., the user's gaze direction 119 over time, user saccadic behavior over time, user eye dilation behavior over time, etc. The one or more sensors 116 may capture audio information including the user's speech and other user-made sounds as well as sounds within the physical environment 100.

One or more sensors, such as one or more sensors 115 on device 105, may identify user information based on proximity or contact with a portion of the user 110. As example, the one or more sensors 115 may capture sensor data that may provide biological information relating to a user's cardiovascular state (e.g., pulse), body temperature, breathing rate, etc.

The one or more sensors 116 or the one or more sensors 115 may capture data from which a user orientation 121 within the physical environment can be determined. In this example, the user orientation 121 corresponds to a direction that a torso of the user 110 is facing.

In some implementations, the content provided by the device 105 and sensor features of device 105 may be provided using components, sensors, or software modules that are sufficiently small in size and efficient with respect to power consumption and usage to fit and otherwise be used in lightweight, battery-powered, wearable products such as wireless ear buds or other ear-mounted devices or head mounted devices (HMDs) such as smart/augmented reality (AR) glasses. Features can be facilitated using a combination of multiple devices. For example, a smart phone (connected wirelessly and interoperating with wearable device(s)) may provide computational resources, connections to cloud or internet services, location services, etc.

In some implementations, data is shared amongst a group of devices to improve user state or environment understanding. For example, device 125 may share information (e.g., images, audio, or other sensor data) corresponding to user 110 or the physical environment 100 (including information about user 130 or user 160) with device 105 so that device 105 can better understand user 110 and physical environment 100.

In some implementations, devices 105, 125, 165 are head mounted devices (HMDs) that present visual or audio content (e.g., extended reality XR content) or have sensors that obtain sensor data (e.g., visual data, sound data, depth data, ambient lighting data, etc.) about the environment 100 or sensor data (e.g., visual data, sound data, depth data, physiological data, etc.) about the users 110, 130, 160. Such information may, subject to user authorizations, permissions, and preferences, be shared amongst the device 105, 125, 165 to enhance the user's experiences on such devices.

In some implementations, the devices 105, 125, 165 obtain physiological data (e.g., EEG amplitude/frequency, pupil modulation, eye gaze saccades, etc.) from the users 110, 130, 160 via one or more sensors that are proximate or in contact with the respective user 110, 130, 160. For example, the device 105 may obtain pupillary data (e.g., eye gaze characteristic data) from an inward facing eye tracking sensor. In some implementations, the devices 105, 125, 165 include additional sensors for obtaining image or other sensor data of the physical environment 100.

In some implementations, the devices 105, 125, 165 are wearable devices such as ear-mounted speaker/microphone devices (e.g., headphones, ear pods, etc.), smart watches, smart bracelets, smart rings, smart/AR glasses, or other head-mounted devices (HMDs). In some implementations, the devices 105, 125, 165 are handheld electronic devices (e.g., smartphones or tablets). In some implementations, the devices 105, 125, 165 are laptop computers or desktop computers. In some implementations, the devices 105, 125, 165 have input devices such as audio command input systems, gesture recognition-based input systems, touchpads or touch-sensitive displays (also known as a “touch screen” or “touch screen display”). In some implementations, multiple devices are used together to provide various features. For example, a smart phone (connected wirelessly and interoperating with wearable device(s)) may provide computational resources, connections to cloud or internet services, location services, etc.

FIG. 1 illustrates an example in which the devices within the physical environment 100 include HMD devices 105, 125, 165. Numerous other types of devices may be used including mobile devices, tablet devices, wearable devices, hand-held devices, personal assistant devices, AI-assistant-based devices, smart speakers, desktop computing devices, menu devices, cash register devices, vending machine devices, juke box devices, or numerous other devices capable of presenting content, capturing sensor data, or communicating with other devices within a system, e.g., via wireless communication. For example, assistance may be provided to a vision impaired person to help the person understand a menu by providing data from the menu to a device being worn by the vision impaired person, e.g., enabling that device to enhance the user's understanding of the menu by providing visual annotations, audible cues, etc.

In some implementations, the devices 105, 125, 165 include eye tracking systems for detecting eye position and eye movements. For example, an eye tracking system may include one or more infrared (IR) light-emitting diodes (LEDs), an eye tracking camera (e.g., near-IR (NIR) camera), and an illumination source (e.g., an NIR light source) that emits light (e.g., NIR light) towards the eyes of the user. Moreover, an illumination source on a device may emit NIR light to illuminate the eyes of the user and the NIR camera may capture images of the eyes of the user. In some implementations, images captured by the eye tracking system may be analyzed to detect position and movements of the eyes of the user, or to detect other information about the eyes such as pupil dilation or pupil diameter. Moreover, the point of gaze estimated from the eye tracking images may enable gaze-based interaction with content shown the device. Additional cameras may be included to capture other areas of the user (e.g., an HMD with a jaw cam to view the user's mouth, a down cam to view the body, an eye cam for tissue around the eye, and the like). These cameras and other sensors can detect motion of the body, or signals of the face modulated by the breathing of the user (e.g., remote PPG).

In some implementations, the devices 105, 125, 165 have graphical user interfaces (GUIs), one or more processors, memory and one or more modules, programs or sets of instructions stored in the memory for performing multiple functions. In some implementations, the users 110, 130, 160 may interact with a GUI through voice commands, finger contacts on a touch-sensitive surface, hand/body gestures, remote control devices, or other user input mechanisms. In some implementations, the functions include viewing/listening to content, image editing, drawing, presenting, word processing, website creating, disk authoring, spreadsheet making, game playing, telephoning, video conferencing, e-mailing, instant messaging, workout support, digital photographing, digital videoing, web browsing, digital music playing, or digital video playing. Executable instructions for performing these functions may be included in a computer readable storage medium or other computer program product configured for execution by one or more processors.

In some implementations, the devices 105, 125, 165 employ various physiological or behavioral sensor, detection, or measurement systems. Detected physiological data may include, but is not limited to, EEG, electrocardiography (ECG), electromyography (EMG), functional near infrared spectroscopy signal (fNIRS), blood pressure, skin conductance, or pupillary response. Detected behavioral data may include, but is not limited to, facial gestures, facial expressions, body gestures, or body language based on image data, voice recognition based on acquired audio signals, etc.

In some implementations, the devices 105, 125, 165 (or other devices) may be communicatively coupled to one or more additional sensors. For example, a sensor (e.g., an EDA sensor) may be communicatively coupled to a device 105, 125, 165 via a wired or wireless connection, and such a sensor may be located on the skin of a user (e.g., on the arm, placed on the hand/fingers of the user, etc.). For example, such a sensor can be utilized for detecting EDA (e.g., skin conductance), heart rate, or other physiological data that utilizes contact with the skin of a user. Moreover, a device 105, 125, 165 (using one or more sensors) may concurrently detect multiple forms of physiological data in order to benefit from synchronous acquisition of physiological data or behavioral data. Moreover, in some implementations, the physiological data or behavioral data represents involuntary data, e.g., responses that are not under conscious control. For example, a pupillary response may represent an involuntary movement. In some implementations, a sensor is placed on the skin as part of a watch device, such as a smart watch.

In some implementations, one or both eyes of a user, including one or both pupils of the user present physiological data in the form of a pupillary response (e.g., eye gaze characteristic data). The pupillary response of the user may result in a varying of the size or diameter of the pupil, via the optic and oculomotor cranial nerve. For example, the pupillary response may include a constriction response (miosis), e.g., a narrowing of the pupil, or a dilation response (mydriasis), e.g., a widening of the pupil. In some implementations, a device may detect patterns of physiological data representing a time-varying pupil diameter. In some implementations, the device may further determine the interpupillary distance (IPD) between a right eye and a left eye of the user.

The user data (e.g., upper facial feature characteristic data, lower facial feature characteristic data, and eye gaze characteristic data, etc.), including information about the position, location, motion, pose, etc., of the head or body of the user, may vary in time and a device 105, 125, 165 (or other devices) may use the user's data to track a user state. In some implementations, the user data includes texture data of the facial features such as eyebrow movement, chin movement, nose movement, cheek movement, etc. For example, when a person (e.g., user 110, 130, 160) performs a facial expression or micro expression associated with lack of familiarity or confusion, the upper and lower facial features can include a plethora of muscle movements that are used to assess the state of the user based on the captured data from sensors.

The physiological data (e.g., eye data, head/body data, etc.) and behavioral data (e.g., voice, facial recognition, etc.) may vary in time and the device may use the physiological data or behavioral data to measure a physiological/behavioral response or the user's attention to object or intention to perform an action. Such information may be used to identify a state of the user with respect to whether the user needs or desires assistance.

Information about such assistance predictions and how a user's own data is used may be provided to a user and the user given the option to opt out of automatic predictions use of their own data and given the option to manually override assistance features. In some implementations, the system is configured to ensure that users' privacy is protected by requiring permissions to be granted before user state is assessed or assistance is enabled.

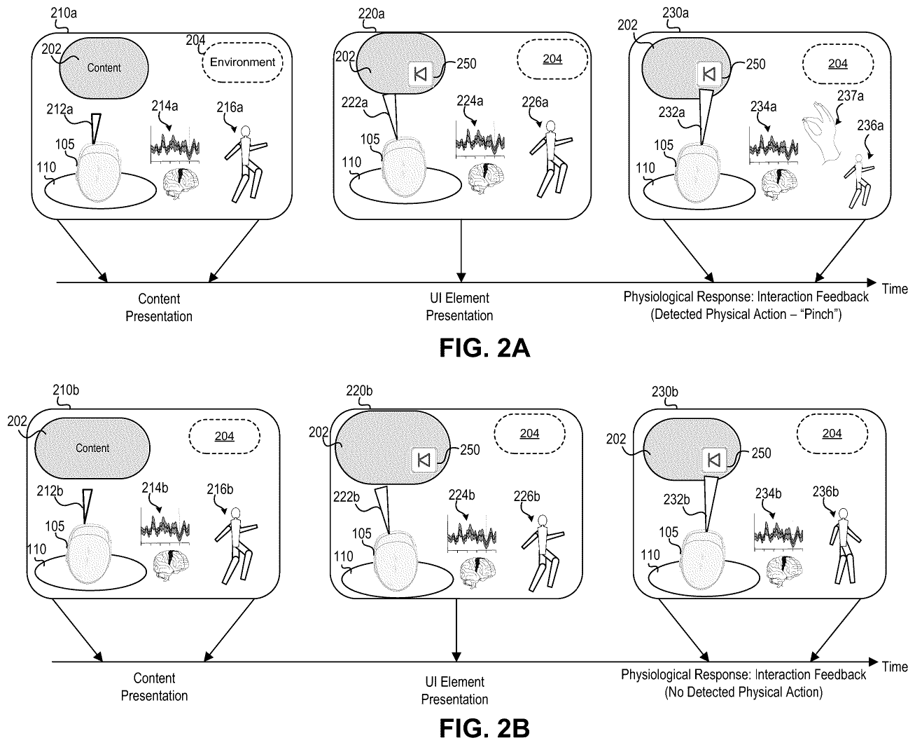

FIGS. 2A and 2B illustrate assessing whether there is an interaction event while viewing content based on physiological data. FIG. 2A illustrates a user (e.g., user 110 wearing device 105 of FIG. 1) being presented with content 202 in an environment 204 during a content presentation where the user, via obtained physiological data, has a physiological response to the content (e.g., the user looks towards portions of the content as detected by eye gaze characteristic data). For example, at content presentation instant 210a, a user is being presented with content 202 that includes visual content (e.g., a video, virtual reality, augmented reality, and the like), and the user's physiologic data such as pupillary data 212a (e.g., eye gaze characteristic data), neurological data 214a (EEG data), body/head movement data 216a is monitored as a baseline. Then, at content presentation instant 220a, while the user pupillary data 222a is engaged (e.g., looking at) content 202, and neurological data 224a (EEG data), body/head movement data 226a is monitored, the content 202 presents interactive element 250. After a segment of time after the user's physiological data is analyzed (e.g., by a physiological data instruction set), as illustrated at content presentation instant 230a, the user's pupillary data 232a, neurological data 234a, and body/head movement data 236a is monitored as the user 110 is focused upon the interactive element 250, as the user interacts with the interactive element 250 (e.g., indicated by the right hand 237a performing a pinching motion to “click” on the icon). Therefore, the content 202 may be updated based on the interaction of the user upon the interactive element 250 (e.g., the user wants to select the virtual icon represented by interactive element 250 by performing a physical act of a pinch). As the user 110 performs the right hand 237a pinch, the neurological signals 234a are collected and analyzed for determining whether the system can predict the physical action based only on the neurological signals.

FIG. 2B illustrates a similar example as FIG. 2A, except that the user is attempting to interact with the interactive element 250 without performing a physical act (e.g., the user is instructed to try and select the virtual icon being presented to him or her by thinking about a pinching motion without moving his or her arm, wrist, or hand). For example, at content presentation instant 210b, a user is being presented with content 202 that includes visual content (e.g., a video), and the user's physiologic data such as pupillary data 212b (e.g., eye gaze characteristic data), neurological data 214b (EEG data), body/head movement data 216b is monitored as a baseline. Then, at content presentation instant 220b, while the user pupillary data 222b is engaged (e.g., looking at) content 202, and neurological data 224b (EEG data), body/head movement data 226b is monitored, the content 202 presents interactive element 250. After a segment of time after the user's physiological data is analyzed (e.g., by a physiological data instruction set), as illustrated at content presentation instant 230b, the user's pupillary data 232a, neurological data 234b, and body/head movement data 236b is monitored as the user 110 is focused upon the interactive element 250, as the user interacts with the interactive element 250 (e.g., the user 110 is thinking of performing a pinching motion to “click” on the icon). Therefore, the content 202 may be updated based on the prediction of an interaction of the user upon the interactive element 250 based on the neurological data 234b (e.g., the user thinks about selecting the virtual icon represented by interactive element 250 by imagining/thinking of performing a physical act of a pinch). As the user 110 imagines/thinks of performing the physical act of a pinch, the neurological signals 234a are collected and analyzed for determining and improving the system predicting a physical action based only on the neurological signals 234b.

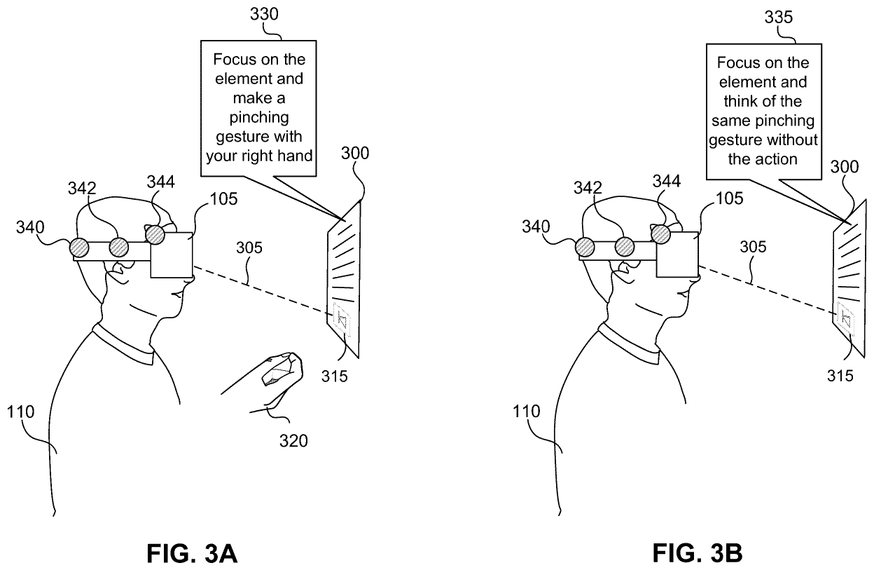

FIGS. 3A and 3B illustrate detecting an interaction event with a cognitive based user interface based on neurological data in accordance with some implementations. FIGS. 3A and 3B illustrate the user 110 wearing device 105, as the user 102 is virtually interacting with a user interface element 315 of a user interface 300 while gazing along gaze direction 305. In particular, FIGS. 3A and 3B illustrate an interaction with user interface 300 as the user is facing the user interface 300. In this example, the user 102 is using device 105 to view and interact with an XR environment that includes the user interface 300. The neurological data may be obtained by the electrodes 340, 342, and 344.

The example of FIG. 3A illustrates the user 110 is shown the interaction element 315 on the user interface 300 and the user is instructed to focus on the interaction element 315 as if they are trying to select the element by a physical act, as illustrated by the notification 330 (e.g., “Focus on the element and make a pinching gesture with your right hand”). For example, instructing the user to look at and reach out towards the interaction element 315 and make a pinch gesture (e.g., as illustrated by right hand 320). The example of FIG. 3B illustrates the user 110 is shown the interaction element 315 on the user interface 300, and the user is instructed to utilize that same focused attention towards the interaction element 315, but without the physical act, as illustrated by the notification 335 (e.g., “Focus on the element and think of the same pinching gesture without the action”). For example, have the user thinking of making the pinch gesture, but not moving his or her hand or arm, etc. The notifications 330, 335 may be visual notifications, audio notifications, or both visual and audio notifications.

In various implementations, the device 105 includes, or is connected to, a neurological sensing system (e.g., EEG monitoring) for capturing neurological signals of the user. As illustrated in FIGS. 3A and 3B, the neurological sensing system may include electrodes 340, 342, and 344. In various implementations, electrode 340, electrode 342, and electrode 344 capture various neurological signals that are received by a receiver at the device 105 either via wireless or direct electrical connections (wired). In various implementations, a controller of the device can determine various neural processes that are mediated through the electrical signals captured by the sensors.

In various implementations, the electrodes 340, 342, and 344 may be located on the outer user-facing surface of a housing of the device 105 (e.g., electrode 340, which contacts the skin/forehead of the user when worn), or the electrodes may be positioned on a head strap or other mounting system for the head worn device 105 (e.g., electrodes 342 and 344, such that when the device 105 is worn the electrodes would contact the head of the user 110). The illustration of FIGS. 3A and 3B is for illustrative purposes and is not meant to be limiting regarding a number or electrodes, and/or the various positioning of the neurological sensing system components (e.g., location of the electrodes). For example, the electrodes of the neurological sensing system may be located on a separately worn device or a separate head strap that could send the various neurological signals to the device 105.

In some implementations, a body tracking algorithm may track arm motion to reduce false positives for identifying a false pinch or another type of movement. For example, arm motion may trigger neural signals that are similar to a pinch. The device 105 may include external facing cameras to capture images (e.g., RGB and/or depth data) of the user's body, and that image data may be used by a body tracking algorithm that can identify periods of rapid arm motion. Thus, a motor intention prediction system can separate out arm motion neural signals to decrease potential false positives for a pinch signal.

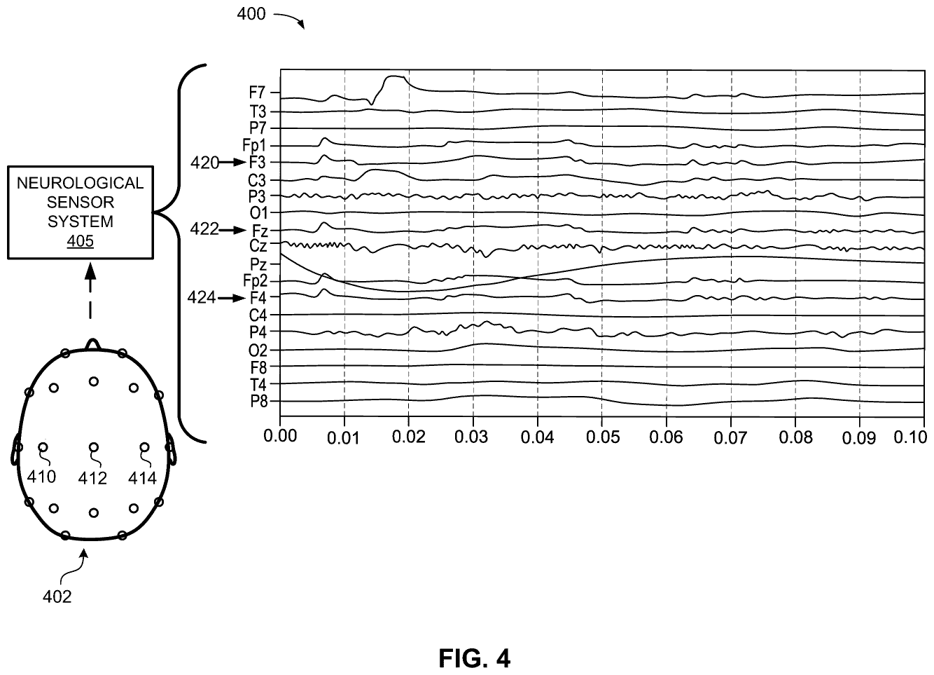

FIG. 4 is an example chart 400 illustrating neurological signals for a neurological sensor system during an experience in accordance with some implementations. In particular, FIG. 4 illustrates an example neurological sensor system 405 that may obtain multiple neurological signals from the multiple neurological sensors (e.g., EEG electrodes) of the EEG system 402. Neural processes may be mediated through electrical signals. The neurological sensor system 405 may include a headset that includes the multiple neurological sensors (e.g., EEG electrodes) of the EEG system 402 that pick up those neurological signals from the brain placed around a head of a user. For example, a headset may include 21 active electrodes that read signals from all over the head of a user.

The chart 400 illustrates data for each sensor of the multiple neurological sensors from the EEG system 402. As illustrated, some neurological signals are quite weak, and are localized to only the motor cortex. For example, in a traditional EEG setup, the electrodes over the central part of the brain (e.g., C3 sensor 410, Cz sensor 412, and C4 sensor 414), may be the best sensors to pick up the sensorimotor rhythm (SMR). However, using an HMD (e.g., a display module and a head support/attachment system, such as a strap), such as device 105, the sensors/electrodes may be located farther back on the head, so that the frontal electrodes (e.g., F3, Fz, and F4) are positioned over the motor cortex. However, the location of the sensors and/or the type of neurological signals received may be obtained from different portions of the head/brain of the user, and the prediction system discussed herein may utilize different combinations of the different type of neurological signals received.

In some implementations, for predicting motor intention of a physical action, such as, inter alia, a pinching gesture, three motor cortex signals may be analyzed. For example, neurological signal 420 (F3 electrode), neurological signal 422 (Fz electrode), and neurological signal 424 (F4 electrode) may be used motor intention prediction according to techniques described herein. For example, three sensors may be a minimum number of neurological signals that are necessary to differentiate the signals (e.g., to determine whether there is a left-handed or right-handed click intention). However, to detect a unilateral click intention, the system may only need to obtain a minimum of two neurological signals.

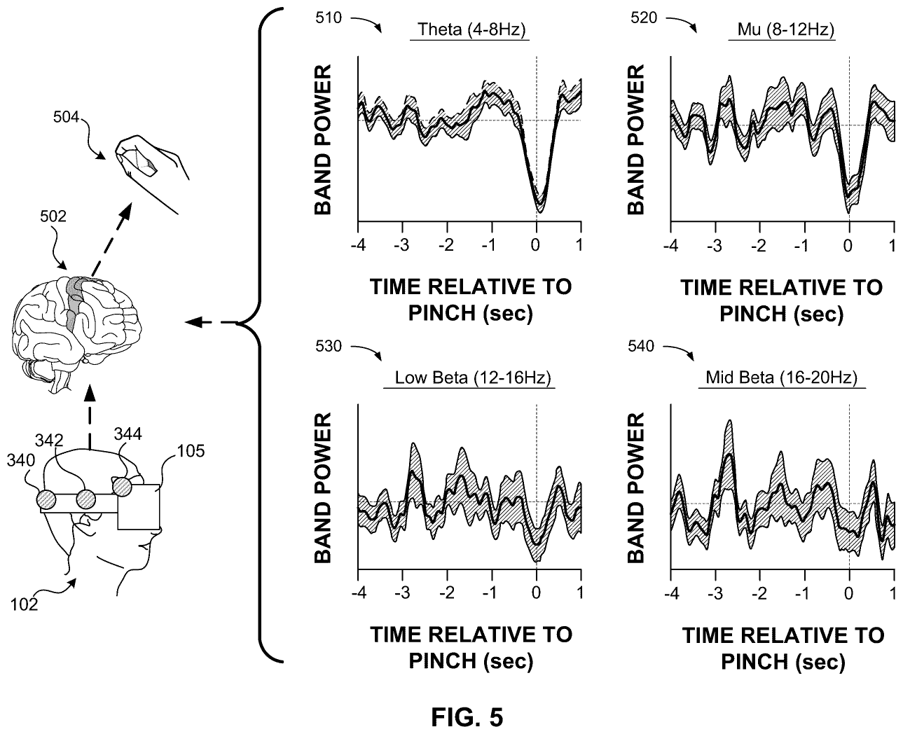

FIG. 5 illustrates obtaining specific neurological band signals while a user is thinking of performing a physical action during an experience in accordance with some implementations. In particular, FIG. 5 illustrates a user 102 wearing a device 105 (e.g., an HMD) that includes neurological sensors (e.g., EEG sensors 340, 342, 344) while the user 102 is thinking of (e.g., a motor cortex area as illustrated by highlighted portion 502) and performing a pinch action with his or her right hand 504. For example, FIG. 5 provides an average of signals for multiple users while each user is instructed to make a physical act, such as a pinch gesture, for a particular band. For example, each graph 510, 520, 530, and 540 illustrates a transformation of a particular neurological signal (e.g., F3 electrode-left motor cortex) into band power, and then lined up a few seconds prior to each physical act (e.g., a pinch) or a thought of making such a physical act (e.g., thinking of making a pinch) and averaged the band powers together.

In particular, graph 510 illustrates the Theta band, a component of the EEG signal that oscillates at 4-8 cycles per second. Graph 520 illustrates the Mu band, a component of the EEG signal that oscillates at 8-12 cycles per second. Graph 530 illustrates the low Beta band, a component of the EEG signal that oscillates at 12-16 cycles per second. Graph 540 illustrates the mid Beta band, a component of the EEG signal that oscillates at 16-20 cycles per second.

For example, each participant maybe shown the interaction element 315 of FIG. 3A and are instructed to focus on the interaction element 315 as if they are trying to select the element by a physical act, such as reaching out and making a pinch gesture (e.g., notification 330:“Focus on the element and make a pinching gesture with your right hand”). Then, the user may be instructed to utilize that same focused attention towards the interaction element 315 without the physical act in order to have the user thinking of making the pinch gesture, but not moving his or her hand or arm, etc. (e.g., notification 335:“Focus on the element and think of the same pinching gesture without the action”). In some implementations, when users make a pinch gesture (or thinks about making a pinch gesture), there may be a decrease in power in each band (e.g., a neurological event).

In some implementations, predicting that the user is thinking of performing the physical act with the portion of the body of the user (e.g., thinking of performing the physical act of the pinch) is based on providing a prediction model (e.g., a neural network) a few different frequency bands from the spectrogram (e.g., graph 500) on which to base a prediction for each user. The spectrogram may consist of multiple time-varying band power signals (as illustrated by graph 500 in FIG. 5), but may further include an entire range from 1-40 Hz in small increments. The small increments for the entire range allows a prediction model to utilize a wider range of EEG activity in making a prediction. In other words, a user-adaptive learning model may utilize different frequency bands for an analysis to determine which frequency band would better predict that a user is thinking of performing the physical act with the portion of the body of the user.

In some implementations, predicting that the user is thinking of performing the physical act with the portion of the body of the user (e.g., thinking of performing the physical act of the pinch) is based on identifying a neurological event with at least one of the neurological signals. For example, as illustrated in each graph 510, 520, 530, 540 there may be an identifiable neurological event, such as a decrease in power in each respective band at time stamp 0 relative to the action (e.g., an actual physical act of the pinch, or thinking of performing the physical act of the pinch). In some implementations, identifying the neurological event with the at least one neurological signal includes determining whether one or more components (e.g., Theta band, Mu Band, Beta, etc.) of the at least one neurological signal includes a change in one or more attributes (power) with respect to a threshold. For example, a prediction model may interpret a 50% decrease in Mu band power as an indicator of an intention to pinch.

In some implementations, the techniques described herein can utilize a training or calibration sequence to adapt to the specific physiological characteristics of the neural signals of a particular user 110. In some implementations, the techniques present the user 110 with a training scenario in which the user 110 is instructed to interact with on-screen items (e.g., interactive objects). By providing the user 110 with a known intent or area of interest (e.g., via instructions), the techniques can record the user's physiological data (e.g., neurological data) and identify a pattern associated with the user's physiological data. In some implementations, the techniques can change a visual characteristic (e.g., a feedback mechanism) associated with content in order to further adapt to the unique physiological characteristics of the user 110. For example, the techniques can direct a user to mentally select a button (e.g., an interactive element) associated with an identified area in the center of the screen on the count of three and record the user's physiological data (e.g., neurological data) to identify a pattern associated with the user's interaction event. Moreover, the techniques can change or alter a visual characteristic associated with the feedback mechanism in order to identify a pattern associated with the user's physiological response to the altered visual characteristic.

In some implementations, the pattern associated with the physiological response of the user 110 is stored in a user profile associated with the user and the user profile can be updated or recalibrated at any time in the future. For example, the user profile could automatically be modified over time during a user experience to provide a more personalized user experience (e.g., a personal educational experience for optimal learning experience while studying). In some implementations, a “click” threshold may be utilized for training a machine learning model. For example, a “click” threshold may be increased or decreased in real-time to maximize true positive events and minimize false positives. Additionally, or alternatively, in some implementations, implicit feedback from the user (e.g., if a sequence of interactions indicates one interaction was an error) may be used to determine true versus false positives in real-time and the “click” threshold may be adapted by the system accordingly. In some implementations, the techniques described herein can utilize a training process or calibration sequence to involve “gamification”, where the user learns to achieve a certain task over time where there is an animation that corresponds to the real-time output of a machine learning model prediction about the probability of click based on the neurological signals (e.g., without performing the physical act such as a pinch). For example, controlling and closing a ring animation, where the ring closes in proportion to the model's predicated click probability.

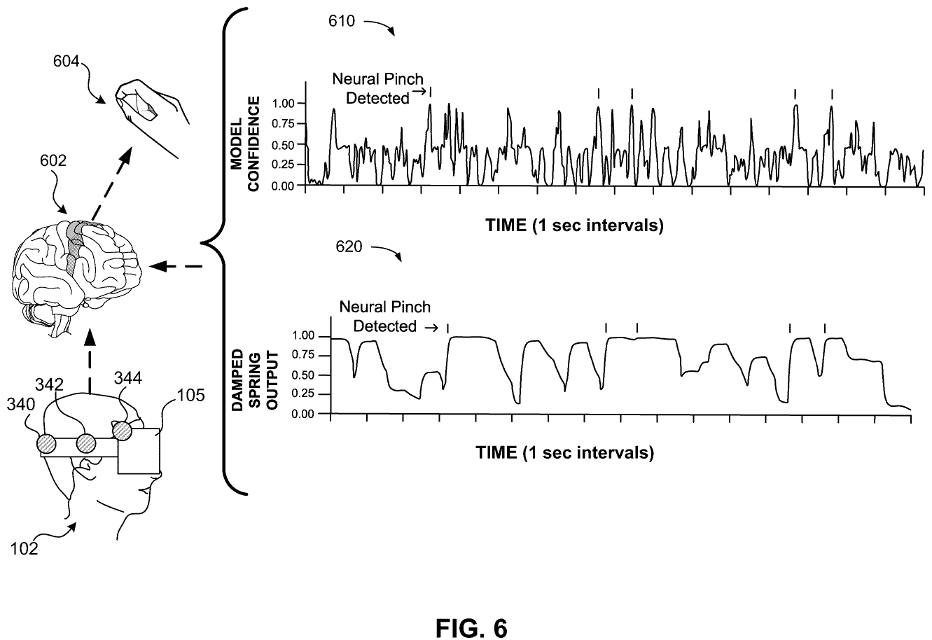

FIG. 6 illustrates damped spring feedback graphs based on neurological signals while a user is thinking of performing a physical action during an experience in accordance with some implementations. In particular, similar to FIG. 5, FIG. 6 illustrates a user 102 wearing a device 105 (e.g., an HMD) that includes neurological sensors (e.g., EEG sensors 340, 342, 344) while the user 102 is thinking of (e.g., a motor cortex area as illustrated by highlighted portion 602) and performing a pinch action with his or her right hand 604. For example, FIG. 6 provides an average of signals for multiple users while each user is instructed to make a physical act, such as a pinch gesture, for a particular band. For example, graph's 610 and 620 each illustrate detecting a pinch based on neurological signals.

In particular, graph 610 illustrates a raw neural “pinch confidence” output of a neurological signal (e.g., an Mu band, a component of the EEG signal that oscillates at 8-12 cycles per second). While graph 620 illustrates applying a damped-spring (smooth spring) dynamic to the same signal from graph 610. In other words, a control policy may utilized that applies a damped-spring transfer function to the raw neural “pinch confidence” output signals. Thus, as illustrated in graph 620 compared to 610, the damped-spring filter produces a slower, more comprehensible feedback curve that users can interpret and learn against.

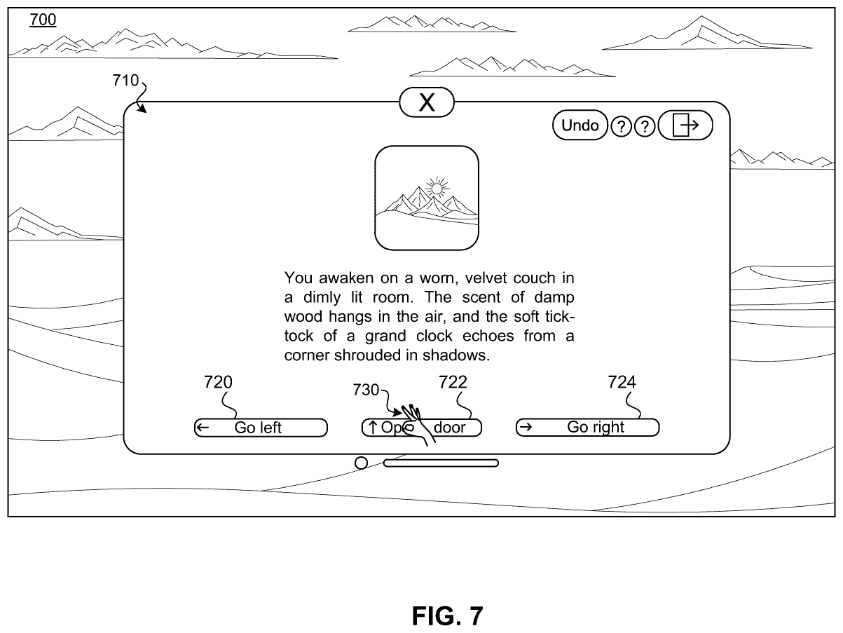

FIG. 7 illustrates detecting an interaction event with a cognitive based user interface and providing a view of a virtual limb based on neurological data in accordance with some implementations. In particular, FIG. 7 illustrates an example of a virtual limb rendering pipeline that includes skeletal animation, coordinate mapping to user interface elements, first-person versus third-person viewpoints, and the like. For example, FIG. 7 provides a view of a 3D environment 700 (e.g., an XR environment), that includes a user interface 710 (e.g., a two-dimensional (2D) user interface to interact with within the XR environment).

The user interface 710 illustrates an example interactive story book application. If the user selects interactive element 720 the story or avatar goes to the left, if the user selects interactive element 724 the story or avatar goes to the right. In this example, the user is selecting to open the door by selecting interactive element 722 using a neurological interface, such as the cognitive user interfaces described herein, by focusing on the interactive element 722 and thinking of a pinching gesture as discussed herein. However, in this example, a virtual limb element 730 is displayed to provide an indication to the user that he or she is trying to select the interactive element 722. In other words, the system renders a virtual representation of the user's hand (or other limb) proximate to a user interface element that is the predicted target. The virtual limb animates toward a pinching motion as the motor-intention confidence rises, thereby leveraging embodiment effects shown to improve brain-computer interface (BCI) performance.

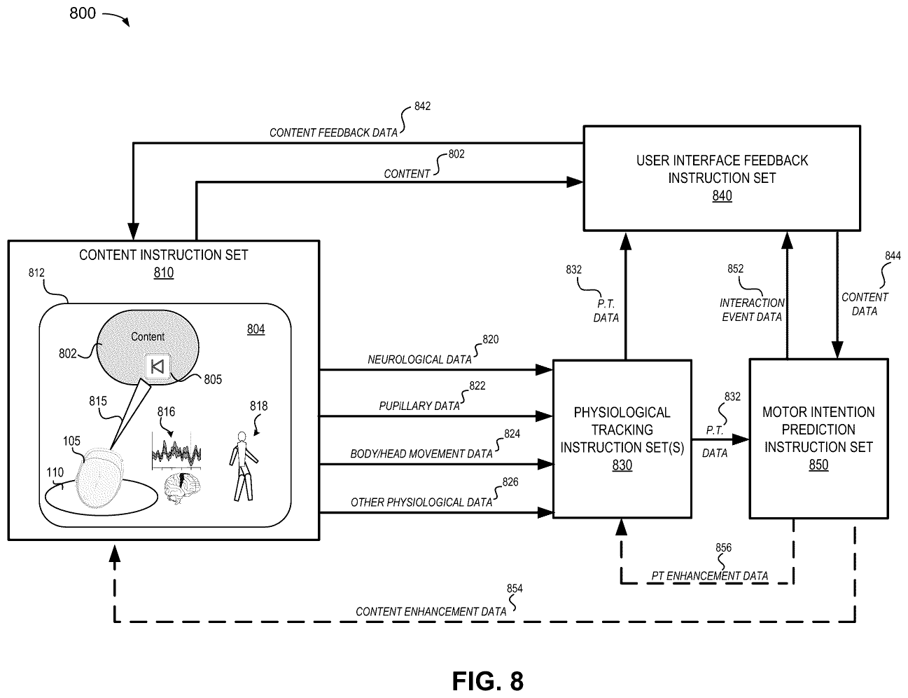

FIG. 8 is a system flow diagram of an example environment 800 in which a motor intention prediction system can assess predictions of a motor intention corresponding to an interaction event based on physiological data (e.g., predicting that the user is thinking of performing a physical act with a portion of a body), according to some implementations. In some implementations, the system flow of the example environment 800 is performed on a device (e.g., device 105 of FIG. 1), such as a mobile device, desktop, laptop, or server device. The content of the example environment 800 can be displayed on a device (e.g., device 105 of FIG. 1) that has a screen for displaying images and/or a screen for viewing stereoscopic images such as an HMD. In some implementations, the system flow of the example environment 800 is performed on processing logic, including hardware, firmware, software, or a combination thereof. In some implementations, the system flow of the example environment 800 is performed on a processor executing code stored in a non-transitory computer-readable medium (e.g., a memory).

The system flow of the example environment 800 acquires and presents content 802 (e.g., video content or a series of image data) to user 110, analyzes the content 802 and/or the environment 804 for context data, obtains physiological data associated with the user during presentation of the content, assesses a user's intent to interact with the interaction element 805 based on the physiological data of the user (e.g., neurological signals) and a prediction of a motor intention, and updates the content based on the interaction event (e.g., if the user 110 focusing his or her attention and thinks of performing a physical act associated with the interaction element 805 (e.g., a pinch) to activate or select the interaction element 805). For example, an motor intention prediction technique described herein predicts, based on obtained physiological data (e.g., neurological signals, such as, inter alia, EEG signals), that the user is thinking of performing a physical act (e.g., a motor intention), and determines the user's intent to interact with the interaction element 805 during an experience (e.g., watching a video) by updating the content that is based on the interaction event (e.g., a notification, auditory signal, an alert, and the like, that alerts the user that they have selected the interaction element 805 during the presentation of content 802).

The example environment 800 includes a content instruction set 810 that is configured with instructions executable by a processor to provide and/or track content 802 for display on a device (e.g., device 105 of FIG. 1). For example, the content instruction set 810 provides content presentation instant 812 that includes content 802 to a user 110 while user is within a physical environment 804 (e.g., a room, outside, etc.). For example, content 802 may include background image(s) and sound data (e.g., a video). The content presentation instant 812 could be an XR experience, or content presentation instant 812 could be a MR experience that includes some CGR content and some images of a physical environment. Alternatively, the user could be wearing a HMD and is looking at a real physical environment either via a live camera view, or the HMD allows a user to look through the display, such as wearing smart glasses that user can see through, but still be presented visual and/or audio cues. During an experience, while a user 110 is viewing the content 802, physiological data may be monitored and tracked and sent to the physiological tracking instruction set 830. For example, neurological signal information 816 (e.g., EEG data) of the user's brain activity (e.g., motor cortex activity) can be monitored and sent as neurological data 820, pupillary information 815 of the user's eyes can be monitored and sent as pupillary data 822, and body and head tracking information 818 of the user's body movement activity can be monitored and sent as body/head movement data 824. Additionally, other physiological data 826 can be monitored and sent as physiological data 814 such as head movement data obtained from an IMU or image data.

The environment 800 further includes a physiological tracking instruction set 830 to track a user's physiological attributes as physiological tracking data 832 using one or more of the techniques discussed herein or as otherwise may be appropriate. For example, the physiological tracking instruction set 830 may acquire physiological data 814 (e.g., neurological signal information 816 (e.g., EEG data)) from the user 110 viewing the content 802 via the device 105. Additionally, or alternatively, a user 110 may be wearing one or more sensors (e.g., such as an EEG electrode, an EDA sensor, heart rate sensor, etc.) that generates sensor data (e.g. IMU or pose data, EEG data, EDA data, heart rate data, and the like) as additional other physiological data 826. Thus, as the content 802 is presented to the user as content presentation instant 812, the physiological data 814 (e.g., neurological signal information 816, pupillary data 815, and the like) and/or other sensor data is sent to the physiological tracking instruction set 830 to track a user's physiological attributes as physiological tracking data 832, using one or more of the techniques discussed herein or as otherwise may be appropriate.

In an example implementation, the environment 800 further includes a user interface feedback instruction set 840 that is configured with instructions executable by a processor to obtain the experience data presented to the user (e.g., content 802), obtain the physiological tracking data 832, and the interaction event data 852, and generates content feedback data 842. For example, the user interface feedback instruction set 840 receives a confirmation of an interaction event, such as a selection of an icon based on determining the user is thinking of performing an action or is actually performing an action, i.e., a pinch, while the user is viewing the presentation of the content 802.

In an example implementation, the environment 800 further includes an motor intention prediction instruction set 850 that is configured with instructions executable by a processor to assess the user's 110 intent to interact with the interaction element 805 (e.g., predicting that the user is thinking of a physical act) based on a physiological response (e.g., brain activity via neurological data 820) using one or more of the techniques discussed herein or as otherwise may be appropriate. For example, predicting an intent of the user 110 to interact with the interaction element 805 may be based on determining that the user 110 is focused on the interaction element 805 (e.g., based on pupillary data 815) and is wanting to “click” on the element by thinking of performing a physical act, such as a pinch. In particular, the motor intention prediction instruction set 850 acquires physiological tracking data 832 from the physiological tracking instruction set 830 and determines the intent of the user 110 to interact with (select) the interaction element 805 during the presentation of the content 802 while the user is watching content 802. In some implementations, the motor intention prediction instruction set 850 can then provide interaction event data 852 (e.g., data that signals that the user selected the interaction element 805) to the user interface feedback instruction set 840 based on the motor intention prediction.

In some implementations, the motor intention prediction instruction set 850 also acquires content data 844 from the user interface feedback instruction set 840 (e.g., scene understanding data) with the physiological tracking data 832 to determine the intent of the user 110 to interact with (select) the interaction element 805 during the presentation of the content 802. For example, the content data 844 may provide a scene analysis that can be used by the motor intention prediction instruction set 850 to understand what the person is looking at, where they are at, etc., and improve the determination of the intent of the user to select the interaction element 805.

In some implementations, the motor intention prediction instruction set 850 may initiate two learning systems to engage with the user 110 using the cognitive based user interface system. First, the motor intention prediction instruction set 850 may learn what the user interface may or may not detect and recommend changes to the content and/or the content element 805 to be updated via the content enhancement data 854 (e.g., update the illumination characteristics of the selectable icon). Second, the motor intention prediction instruction set 850 may enhance a neural signal based on the acquired physiological tracking data 832 and content data 844 and provide physiological tracking enhancement data 856 to the physiological tracking instruction set 830 (e.g., enhance the accuracy and sensitivity of a neural pinch signal for each user). In other words, the quality of the received neural signals may vary between user and/or between each experience, and the motor intention prediction instruction set 850 may customize each session and/or for each user via the physiological tracking enhancement data 856 (e.g., biofeedback training for each user). The customization may be based on fine tuning a neural model for enhanced accuracy based on the brain activity and user interface feedback in order to learn what the cognitive based system can detect and how to customize the enhanced neural signals. In some implementations, the motor intention prediction instruction set 850 is trained and updated while the user 110 is interacting with the system, and the motor intention prediction instruction set 850 is then improved with the user 110 over time (e.g., a biofeedback learning system).

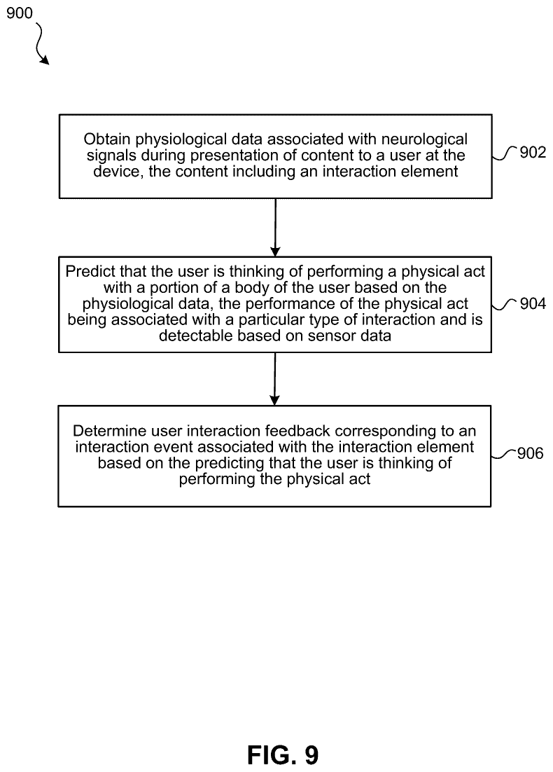

FIG. 9 is a flowchart illustrating an exemplary method 900. In some implementations, a device such as device 105 (FIG. 1) performs the techniques of method 900 to determine user interaction feedback corresponding to an interaction event by predicting physical act intentions based on neurological signals. For example, the method 900 may identify that, during a particular segment of an experience, the gaze characteristics (e.g., pupillary data, such as, inter alia, pupil dilation vs. constriction, stable gaze direction, velocity of pupil movements, and the like) corresponds to a user focusing on (looking at) a particular icon or user interface element (referred to herein as an “interactive element”) and based on the neurological data (e.g., EEG data) a prediction can be assessed whether a user is thinking of performing a physical act that is associated with a particular type of interaction (e.g., a pinch-based selection). For example, a user may direct his or her attention to an icon (or a particular feature/attribute of an icon, such as a bright feature) in order to initiate a “click” or other interaction. This prediction of an intended interaction based on neurological signals (without using a physical movement of a body part) may be used as a user interface selection tool, device wake-up signal, etc., and might be combined with other eye or touch-based mechanisms to improve SNR, robustness, and response time. In other words, someone who may be unable to move his or her arms/hands (e.g., a person who is a quadriplegic) can look at an icon, think about making a physical act, such as a pinch, and interact with a user interface.

In some implementations, the techniques of method 900 are performed on a mobile device, desktop, laptop, HMD, or server device. In some implementations, the method 900 is performed on processing logic, including hardware, firmware, software, or a combination thereof. In some implementations, the method 900 is performed on a processor executing code stored in a non-transitory computer-readable medium (e.g., a memory).

At block 902, the method 900 obtains physiological data associated with neurological signals during presentation of content to a user at the device, the content including an interaction element. For example, the neurological signals may include brain activity, EEG signals, and the like. The interaction element may include a selectable user interface element (e.g., an icon).

In some implementations, obtaining physiological data includes EEG amplitude/frequency, pupil modulation, eye gaze saccades, head movements, body movements (e.g., hand and/or arm movements), and the like, from which neurological response, pupil response, gaze direction/movement, and the like, may be determined. In some implementations, obtaining physiological data (e.g., neurological data, pupillary data, etc.) is associated with a gaze of a user that may involve obtaining images of the eye or electrooculography signal (EOG) data from which gaze direction and/or movement can be determined. In some implementations, the physiological data includes at least one of skin temperature, respiration, photoplethysmogram (PPG), electrodermal activity (EDA), eye gaze tracking, and pupillary movement that is associated with the user. In some implementations, obtaining physiological data includes body movements and/or head movements (e.g., obtained from an IMU or from image sensor data).

Some implementations obtain physiological data and other user information to help improve a user experience. In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user will have the ability to access and otherwise find out anything that the system has collected or determined about him or her. User data is stored securely on the user's device. User data that is used as input to a machine learning model is stored securely on the user's device, for example, to ensure the user's privacy. The user's device may have a secure storage area, e.g., a secure enclave, for securing certain user information, e.g., data from image and other sensors that is used for face identification, face identification, or biometric identification. The user data associated with the user's body and/or attentive state may be stored in such a secure enclave, restricting access to the user data and restricting transmission of the user data to other devices to ensure that user data is kept securely on the user's device. User data may be prohibited from leaving the user's device and may be used only in machine learning models and other processes on the user's device.

At block 904, the method 900 predicts that the user is thinking of performing a physical act with a portion of a body of the user based on the physiological data, the performance of the physical act being associated with a particular type of interaction and is detectable based on sensor data. For example, the method 900 may predict a particular type of interaction (e.g., a pinch-based selection) of an icon based on EEG data, or other neurological signals. In other words, predicting that the user is imagining/thinking of a type of act (pinch) associated with that type of interaction (e.g., thinking about making a pinch based motion with a hand). In an exemplary implementation, the neurological signals include a minimum of three EEG signals (e.g. left, right, and a ground signal).

In some implementations, predicting that the user is thinking of performing the physical act is based on recognizing (e.g., via a machine learning model or another algorithm) that a pattern exhibited in the neurological signals of the physiological data (e.g., EEG data) is indicative of the user thinking of performing the physical act.

At block 906, the method 900 determines user interaction feedback corresponding to an interaction event associated with the interaction element based on the predicting that the user is thinking of performing the physical act. For example, initiating an action based on determining the user is imagining/thinking of a type of act (e.g., a pinch) associated with that type of interaction. In some implementations, there may be machine learned feedback data, a learning process in the background, and/or a guided training process to learn how “think” or “imagine” of performing the physical act without making the physical act. In some implementations, a cognitive-based user interface can continuously learn in a closed feedback loop process to improve and enhance a neural pinch signal and customize the feedback loop per each user (e.g., each individual person may exhibit stronger or weaker indicators of a particular thought of a physical act) and/or customize to each particular experience (e.g., weaker signal detection based on, inter alia, placement/position of electrodes, skin/hair conditions of the user, electrode degradation, and the like).

In some implementations, the method 900 further includes, in response to determining, based on the sensor data, that the physical act associated with the particular type of interaction is performed, obtaining additional physiological data associated with neurological signals, and updating a prediction model associated with the user performing the physical act associated with the particular type of interaction based on the additional physiological data. For example, a physical act is performed, and the EEG data confirms the user intentions to perform a “click” and allows for quicker response in a subsequent act (e.g., the cognitive-based user interface improves on the accuracy of detecting and predicting a pinch intent action).

In some implementations, the method 900 further includes, in response to determining, based on the sensor data, that the physical act associated with the particular type of interaction is not performed while the user is thinking of performing the physical act, obtaining additional physiological data associated with neurological signals, and updating a prediction model associated with the user performing the physical act associated with the particular type of interaction based on the additional physiological data. For example, a physical act is not performed, and the EEG data is a proxy for actual movement providing accessibility functionality for those who can't pinch (e.g., the cognitive-based user interface predicted a pinch intent action without the user performing the physical act).

In some implementations, predicting that the user is thinking of performing the physical act with the portion of the body of the user (e.g., thinking of performing the physical act of the pinch) is based on identifying a neurological event with at least one of the neurological signals. For example, as illustrated in FIG. 5, for each graph 510, 520, 530, 540, there may be an identifiable neurological event, such as a decrease in power in each respective band at time stamp 0 relative to the action (e.g., an actual physical act of the pinch, or thinking of performing the physical act of the pinch). In some implementations, identifying the neurological event with the at least one neurological signal includes determining whether one or more components (e.g., Theta band, Mu Band, Beta, etc.) of the at least one neurological signal includes a change in one or more attributes (power) with respect to a threshold. For example, a prediction model may interpret a 50% decrease in Mu band power as an indicator of an intention to pinch.

In some implementations, for predicting motor intention of a physical action, such as, inter alia, a pinching gesture, at a minimum, the three main motor cortex signals may be analyzed. In some implementations, the device includes at least three neurological sensors configured to obtain the physiological data associated with neurological signals corresponding to main motor cortex signals (e.g., EEG electrodes for monitoring three main motor cortex signals). For example, neurological signal 420 (e.g., an F3 electrode), neurological signal 422 (e.g., an Fz electrode), and neurological signal 424 (e.g., an F4 electrode) may be used motor intention prediction. For example, three sensors may be a minimum number of neurological signals that are necessary to differentiate the signals (e.g., to determine whether there is a left-handed or right-handed click intention). However, to detect a unilateral click intention, the system may only need to obtain a minimum of two neurological signals.

In some implementations, each sensor of the at least three neurological sensors are positioned at different regions of the device. In some implementations, the different regions of the device that each of the at least three neurological sensors are positioned includes a support element (e.g., a head strap) of the device, on a user facing surface adjacent to a lens of the device, via an additional support element coupled to the device, or a combination thereof. For example, as illustrated in FIGS. 3A and 3B, the electrodes 340, 342, and 344 may be located on the outer user-facing surface of a housing of the device 105 (e.g., electrode 340, which contacts the skin/forehead of the user when worn), or the electrodes may be positioned on a head strap or other mounting system for the head worn device 105 (e.g., electrodes 342 and 344, such that when the device 105 is worn the electrodes would contact the head of the user 110). Additionally, or alternatively, in some implementations, in addition to placing sensors on support element such as a top strap, a back strap may be used to provide supporting data for the neurological sensors. For example, signals from the motor cortex generated in the alpha and beta frequency range may be separated from alpha and beta frequency activity originating in the visual cortex (e.g., same frequency but unrelated to motor processes).

In various implementations, predicting that the user is thinking of performing a physical act may be based on determining a physiological response corresponding to the user directing attention to a region of the interaction element based on different illumination characteristics of the interaction element (e.g., pupillary response characteristics such as pupil constriction when attending to a bright feature of an icon), and then thinking of performing a physical act while directing attention to the interaction element. For example, pupillary response characteristics may include measuring a variability of the pupillary radius. In some implementations, variability may be measured based on range, variance, and/or standard deviation.

In some implementations, the method 900 further includes, prior to presenting the user interaction feedback, filtering a prediction value indicative of a motor intention through a damped-spring control function to generate a smoothed feedback signal. In some implementations, the smoothed feedback signal is based on at least one of a visual, haptic, and an auditory output presented to the user. For example, graph 620 of FIG. 6 illustrates applying a damped-spring (smooth spring) dynamic to the same signal from graph 610. In other words, a control policy may utilized that applies a damped-spring transfer function to the raw neural “pinch confidence” output signals. Thus, as illustrated in graph 620 compared to 610, the damped-spring filter produces a slower, more comprehensible feedback curve that users can interpret and learn against.

In some implementations, the user interaction feedback includes rendering a virtual representation of a portion of the user (e.g., a virtual limb), the representation being animated toward the physical act in proportion to a confidence that the user is thinking of performing the physical act. For example, as illustrated in FIG. 7, a virtual limb element 730 is displayed to provide an indication to the user that he or she is trying to select the interactive element 722. In other words, the system renders a virtual representation of the user's hand (or other limb) proximate to a user interface element that is the predicted target. The virtual limb animates toward a pinching motion as the motor-intention confidence rises, thereby leveraging embodiment effects shown to improve brain-computer interface (BCI) performance. In some implementations, the virtual representation is displayed adjacent to an interaction element predicted to be an intended target. In some implementations, updating the prediction model includes detecting a change in the motor-cortex signal after presentation of the virtual representation and adjusting model parameters based on the detected change.

In some implementations, the method 900 further includes obtaining additional physiological data associated with body movements (e.g., body tracking data), and updating a prediction model associated with predicting that the user is thinking of performing the physical act associated with the particular type of interaction based on the additional physiological data (e.g., a user's body movements may be tracked with cameras and may also be used for prediction). For example, a body tracking algorithm may track arm motion to reduce false positives for identifying a false pinch or another type of movement. For example, arm motion may trigger neural signals that are similar to a pinch. The device 105 may include external facing cameras to capture images (e.g., RGB and/or depth data) of the user's body, and that image data may be used by a body tracking algorithm that can identify periods of rapid arm motion. Thus, a motor intention prediction system can separate out arm motion neural signals to decrease potential false positives for a pinch signal.

In some implementations, determining the neurological response during the presentation of the interaction element is based on determining a variability of the neurological response to a threshold. An example threshold limit for the variability of the neurological response may be based on a machine learning model output. For example, if the machine learning model takes the physiological data as input and outputs a probability of click intent (e.g., 70%), then a determination may be made that any neurological response causing a probability under 70% is no click, while a neurological response leading to a machine learning model output at or above 70% is a click. The same thresholding may be applied to a pupillary response.

Another type of threshold could be an outlier detection, e.g., if the neurological response, pupillary response, or other physiological data changes beyond an accepted range, and that data may be rejected and considered as noise. Likewise, if response changes are so small that the system would have low confidence in measuring such a small change, the system might also reject that data as noise.

In some implementations, the user interaction feedback corresponding to an interaction event associated with the interaction element is classified using a machine learning technique. In some implementations, a machine learning algorithm may be determined based predicting a “click” or “no click” for each time point based on the presence of a prediction of a motor intention (e.g., assessed that a user is thinking of performing a physical act that is associated with a particular type of interaction, e.g., a pinch-based selection). In some implementations, determining an interaction event includes determining scene-induced pupil response variation characteristics for the interaction element, and determining the interaction event during the presentation of the interaction element based on the scene-induced pupil response variation characteristics for the regions of the interaction element. The pupillary response may be a direction of the pupillary response, a velocity of the pupillary response, or pupillary fixations (e.g., derived from eye gaze dynamics and saccade characteristics). For example, when a user's gaze intersects with a user interface element (e.g., interaction element 315 of FIG. 3A), and there is a determination of a prediction of a physical act (e.g., a pinch-based selection), a machine learning protocol can predict a “click” or “no click” for each time point, based on the presence of a prediction of a motor intention response. Therefore, in some implementations, the interaction event may be classified using a machine learning technique based on the pupil response and/or the neurological response (e.g., a machine learning “click” model).

In some implementations, the machine learning model is a neural network (e.g., an artificial neural network), decision tree, support vector machine, Bayesian network, or the like. These labels may be collected from the user beforehand, or from a population of people beforehand, and fine-tuned later on individual users. Creating this labeled data may require many users going through an experience (e.g., a meditation experience) where the users listen to natural sounds with intermixed natural-probes (e.g., an auditory stimulus) and then randomly are asked how focused or relaxed they were (e.g., interaction event) shortly after a probe was presented. The answers to these questions can generate a label for the time prior to the question and a deep neural network or deep long short term memory (LSTM) network might learn a combination of features specific to that user or task given those labels (e.g., low interaction event, high interaction event, etc.).

In some implementations, the method 900 further includes adjusting content in response to determining the interaction event (e.g., the user performing a physical act or imagining/thinking of performing the physical act). For example, as illustrated in the system flow diagram of environment 600 of FIG. 6, when is determined that user's intent is to “click” (e.g., interact/focus) on the interaction element 605, the motor intention prediction instruction set 650 provides interaction event data 652 to the content instruction set 610 to update the content (e.g., change the content based on the selection of the icon—interaction element 605).

In some implementations, the techniques described herein obtain physiological data (e.g., pupillary data, EEG amplitude/frequency data, pupil modulation, eye gaze saccades, head movements, etc.) from the user based on identifying typical interactions of the user with the experience. For example, the techniques may determine that a variability of brain activity and eye gaze characteristic of the user correlates with an interaction with the experience. Additionally, the techniques described herein may then adjust a visual characteristic of the experience, or adjust/change a sound associated with the interaction element, to enhance physiological response data associated with future interactions with the experience and/or the interaction element presented within the experience. Moreover, in some implementations, changing an interaction element after the user interacts with, or is predicted to interact with, the experience informs the physiological response of the user in subsequent interactions with the interaction element or a particular segment of the experience. For example, the user may present an anticipatory physiological response associated with the change within the interaction element (e.g., a change in illuminance of the interaction element). Thus, in some implementations, the technique identifies a prediction of an intent of the user to perform a physical act to interact with the interaction element based on an anticipatory physiological response. For example, the technique may adapt or train an instruction set by capturing or storing physiological data of the user based on the interaction of the user with the experience, and may detect a future intention of the user to interact with the experience by identifying a physiological response of the user in anticipation of the presentation of the enhanced/updated interaction element.

In some implementations, customization of the experience could be controlled by the user. For example, a user could select the experience he or she desires, such as he or she can choose the ambience, background scene, music, etc. Additionally, the user could alter the threshold of selecting the interactive element. For example, the user can customize the sensitivity of triggering the interactive element based on prior experience of a session. For example, a user may desire to not have as many notifications and allow some mind wandering (e.g., eye position deviations) or some body/arm movements before an interactive element is triggered. Thus, particular experiences can be customized on triggering a threshold when higher criteria is met. For example, a user may have to look at a particular interactive element for longer (or shorter) to toggle the interactive element (e.g. shakes or flashes) and/or a longer (or shorter) threshold (e.g., two or more seconds) after the toggle to actually select the interactive element.

In some aspects, the method 900 determines a context of the experience based on sensor data of the environment. For example, determining a context may involve using computer vision to generate a scene understanding of the visual and/or auditory attributes of the environment—where is the user, what is the user doing, what objects are nearby. Additionally, a scene understanding of the content presented to the user could be generated that includes the visual and/or auditory attributes of what the user was watching.

In some aspects, different contexts of the content presented and the environment are analyzed to determine where the user is, what the user is doing, what objects or people are nearby in the environment or within the content, what the user did earlier (e.g., meditated in the morning). Additionally, context analysis may include image analysis (semantic segmentation), audio analysis (jarring sounds), location sensors (where user is), motion sensors (fast moving vehicle), and even access other user data (e.g., a user's calendar). In an exemplary implementation, the method 900 may further include determining the context of the experience by generating a scene understanding of the environment based on the sensor data of the environment, the scene understanding including visual or auditory attributes of the environment, and determining the context of the experience based on the scene understanding of the environment.

In some implementations, the sensor data includes image data, and generating the scene understanding is based at least on performing semantic segmentation of the image data and detecting one or more objects within the environment based on the semantic segmentation. In some implementations, determining the context of the experience includes determining an activity of the user based on the scene understanding of the environment. In some implementations, the sensor data includes location data of the user, and determining the context of the experience includes determining a location of the user within the environment based on the location data.

In some implementations, determining the context of the experience includes determining an activity of the user based on a user's schedule. For example, the system may access a user's calendar to determine if a particular event is occurring when the particular interaction event is assessed. For example, different applications may include different interaction elements to be provided to the user to select via his or her neurological data and/or pupillary response (eye gaze characteristics).

In some implementations, one or more pupillary or EEG characteristics may be determined, aggregated, and used to classify the prediction of the user's intent to perform a physical act to determine an interaction event occurrence using statistical or machine learning techniques. In some implementations, the physiological data is classified based on comparing the variability of the physiological data to a threshold.

In some implementations, the method 900 further includes adjusting content corresponding to the experience based on the interaction event (e.g., customized to the interaction event). For example, content recommendation for a content developer can be provided based on determining interaction events during the presented experience and changes of the experience or content presented therein. For example, the user may focus well (e.g., imagine or think about performing the pinch better) when particular types of content are provided. In some implementations, the method 900 may further include identifying content based on similarity of the content to the experience, and providing a recommendation of the content to the user based on determining that the user has the interaction event during the experience (e.g., mind wandering). In some implementations, the method 900 may further include customizing content included in the experience based on the interaction event (e.g., breaking the content into smaller pieces).

In some implementations, an estimator or statistical learning method is used to better understand or make predictions about the physiological data (e.g., pupillary data characteristics, head movements, etc.). For example, statistics for pupillary data may be estimated by sampling a dataset with replacement data (e.g., a bootstrap method).

In some implementations, the techniques could be trained on many sets of user physiological data and then adapted to each user individually. For example, content creators can customize an experience (e.g., an instructional video) based on the user physiological data, such as a user may require background music, different ambient lighting for learning, or require more or less audio or visual cues to continue to maintain an attention state that is better suited for predicting a motor intention of the user..

In some implementations, the techniques described herein can account for real-world environment 100 of the user 110 (e.g., visual qualities such as luminance, contrast, semantic context) in its evaluation of how much to modulate or adjust the presented content or interactive elements to enhance the physiological response (e.g., pupillary response) of the user 110 to a visual characteristic (e.g., interactive elements).

In some implementations, the techniques described herein can utilize a training or calibration sequence to adapt to the specific physiological characteristics of a particular user 110, as described herein with reference to FIG. 6. In some implementations, the techniques present the user 110 with a training scenario in which the user 110 is instructed to interact with on-screen items (e.g., interactive objects). By providing the user 110 with a known intent or area of interest (e.g., via instructions), the techniques can record the user's physiological data (e.g., neurological data, pupillary data, body/head movement data, etc.) and identify a pattern associated with the user's physiological data. In some implementations, the techniques can change a visual characteristic (e.g., a feedback mechanism) associated with content in order to further adapt to the unique physiological characteristics of the user 110. For example, the techniques can direct a user to mentally select a button (e.g., an interactive element) associated with an identified area in the center of the screen on the count of three and record the user's physiological data (e.g., neurological data, pupillary data, body/head movement data, etc.) to identify a pattern associated with the user's interaction event. Moreover, the techniques can change or alter a visual characteristic associated with the feedback mechanism in order to identify a pattern associated with the user's physiological response to the altered visual characteristic. In some implementations, the pattern associated with the physiological response of the user 110 is stored in a user profile associated with the user and the user profile can be updated or recalibrated at any time in the future. For example, the user profile could automatically be modified over time during a user experience to provide a more personalized user experience (e.g., a personal educational experience for optimal learning experience while studying).

In some implementations, a machine learning model (e.g., a trained neural network) is applied to identify patterns in physiological data (e.g., neurological signals), including identification of physiological responses to presentation of content during a particular experience (e.g., education, meditation, instructional, etc.). Moreover, the machine learning model may be used to match the patterns with learned patterns corresponding to indications of interest or intent of the user 110 to interact with the interaction element. In some implementations, the techniques described herein may learn patterns specific to the particular user 110. For example, the techniques may learn from determining that a peak pattern represents an indication of interest or intent of the user 110 in response to a particular visual characteristic within the content and use this information to subsequently identify a similar peak pattern as another indication of interest or intent of the user 110. Such learning can take into account the user's relative interactions with multiple visual characteristics, in order to further adjust the visual characteristic and enhance the user's physiological response to the experience and the presented content (e.g., focusing on particular areas of content versus other distracting areas).

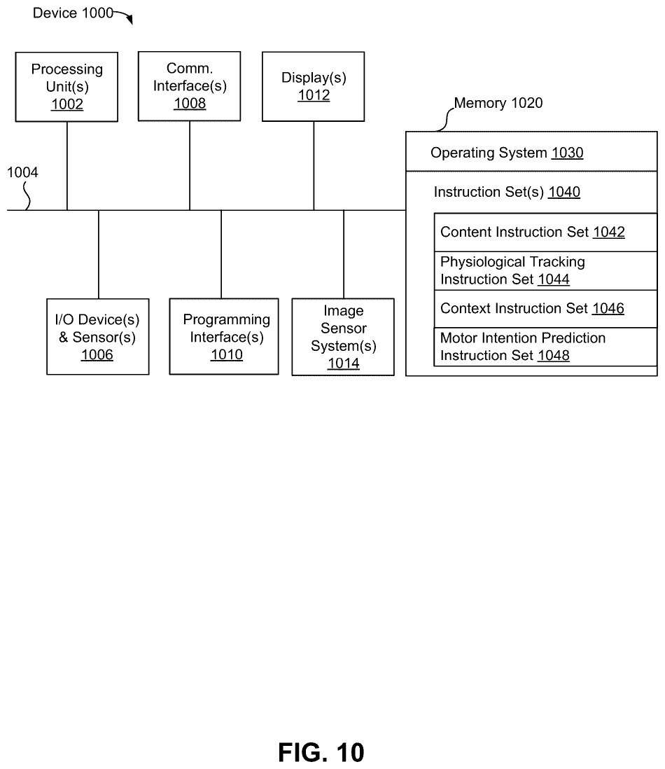

FIG. 10 is a block diagram of an example device 1000. Device 1000 illustrates an exemplary device configuration. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 105 includes one or more processing units 1002 (e.g., microprocessors, ASICs, FPGAs, GPUs, CPUs, processing cores, and/or the like), one or more input/output (I/O) devices and sensors 1006, one or more communication interfaces 1008 (e.g., USB, FIREWIRE, THUNDERBOLT, IEEE 802.3x, IEEE 802.11x, IEEE 802.16x, GSM, CDMA, TDMA, GPS, IR, BLUETOOTH, ZIGBEE, SPI, I2C, and/or the like type interface), one or more programming (e.g., I/O) interfaces 1010, one or more displays 1012, one or more interior and/or exterior facing image sensor systems 1014, a memory 1020, and one or more communication buses 1004 for interconnecting these and various other components.

In some implementations, the one or more communication buses 1004 include circuitry that interconnects and controls communications between system components. In some implementations, the one or more I/O devices and sensors 1006 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), and/or the like.

In some implementations, the one or more displays 1012 are configured to present a view of a physical environment or a graphical environment to the user. In some implementations, the one or more displays 1012 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays 1012 correspond to diffractive, reflective, polarized, holographic, etc. waveguide displays. In one example, the device 1000 includes a single display. In another example, the device 1000 includes a display for each eye of the user.

In some implementations, the one or more image sensor systems 1014 are configured to obtain image data that corresponds to at least a portion of a physical environment (e.g., physical environment 100 of FIG. 1). For example, the one or more image sensor systems 1014 include one or more RGB cameras (e.g., with a complimentary metal-oxide-semiconductor (CMOS) image sensor or a charge-coupled device (CCD) image sensor), monochrome cameras, IR cameras, depth cameras, event-based cameras, and/or the like. In various implementations, the one or more image sensor systems 1014 further include illumination sources that emit light, such as a flash. In various implementations, the one or more image sensor systems 1014 further include an on-camera image signal processor (ISP) configured to execute a plurality of processing operations on the image data.

The memory 1020 includes high-speed random-access memory, such as DRAM, SRAM, DDR RAM, or other random-access solid-state memory devices. In some implementations, the memory 1020 includes non-volatile memory, such as one or more magnetic disk storage devices, optical disk storage devices, flash memory devices, or other non-volatile solid-state storage devices. The memory 1020 optionally includes one or more storage devices remotely located from the one or more processing units 1002. The memory 1020 includes a non-transitory computer readable storage medium.

In some implementations, the memory 1020 or the non-transitory computer readable storage medium of the memory 1020 stores an optional operating system 1030 and one or more instruction set(s) 1040. The operating system 1030 includes procedures for handling various basic system services and for performing hardware dependent tasks. In some implementations, the instruction set(s) 1040 include executable software defined by binary information stored in the form of electrical charge. In some implementations, the instruction set(s) 1040 are software that is executable by the one or more processing units 1002 to carry out one or more of the techniques described herein.

The instruction set(s) 1040 include a content instruction set 1042, a physiological tracking instruction set 1044, a context instruction set 1046, and an motor intention prediction instruction set 1048. The instruction set(s) 1040 may be embodied a single software executable or multiple software executables.

In some implementations, the content instruction set 1042 is executable by the processing unit(s) 1002 to provide and/or track content for display on a device. The content instruction set 1042 may be configured to monitor and track the content over time (e.g., during an experience such as an education session) and/or to identify change events that occur within the content. In some implementations, the content instruction set 1042 may be configured to inject change events into content (e.g., feedback mechanisms) using one or more of the techniques discussed herein or as otherwise may be appropriate. To these ends, in various implementations, the instruction includes instructions and/or logic therefor, and heuristics and metadata therefor.

In some implementations, the physiological tracking instruction set 1044 is executable by the processing unit(s) 1002 to track a user's physiological attributes (e.g., EEG amplitude/frequency, pupil modulation, eye gaze saccades, body/head movement data, etc.) using one or more of the techniques discussed herein or as otherwise may be appropriate. To these ends, in various implementations, the instruction includes instructions and/or logic therefor, and heuristics and metadata therefor.

In some implementations, the context instruction set 1046 is executable by the processing unit(s) 1002 to determine a context of the experience and/or the environment (e.g., create a scene understanding to determine the objects or people in the content or in the environment, where the user is, what the user is watching, etc.) using one or more of the techniques discussed herein (e.g., object detection, facial recognition, etc.) or as otherwise may be appropriate. To these ends, in various implementations, the instruction includes instructions and/or logic therefor, and heuristics and metadata therefor.

In some implementations, the motor intention prediction instruction set 1048 is executable by the processing unit(s) 1002 to predict an intent of the user to interact with an interaction element (e.g., an interaction event determination) based on physiological data associated with neurological signals(e.g., EEG data) using one or more of the techniques discussed herein or as otherwise may be appropriate. To these ends, in various implementations, the instruction includes instructions and/or logic therefor, and heuristics and metadata therefor.

Although the instruction set(s) 1040 are shown as residing on a single device, it should be understood that in other implementations, any combination of the elements may be located in separate computing devices. Moreover, FIG. 10 is intended more as functional description of the various features which are present in a particular implementation as opposed to a structural schematic of the implementations described herein. As recognized by those of ordinary skill in the art, items shown separately could be combined and some items could be separated. The actual number of instructions sets and how features are allocated among them may vary from one implementation to another and may depend in part on the particular combination of hardware, software, and/or firmware chosen for a particular implementation.

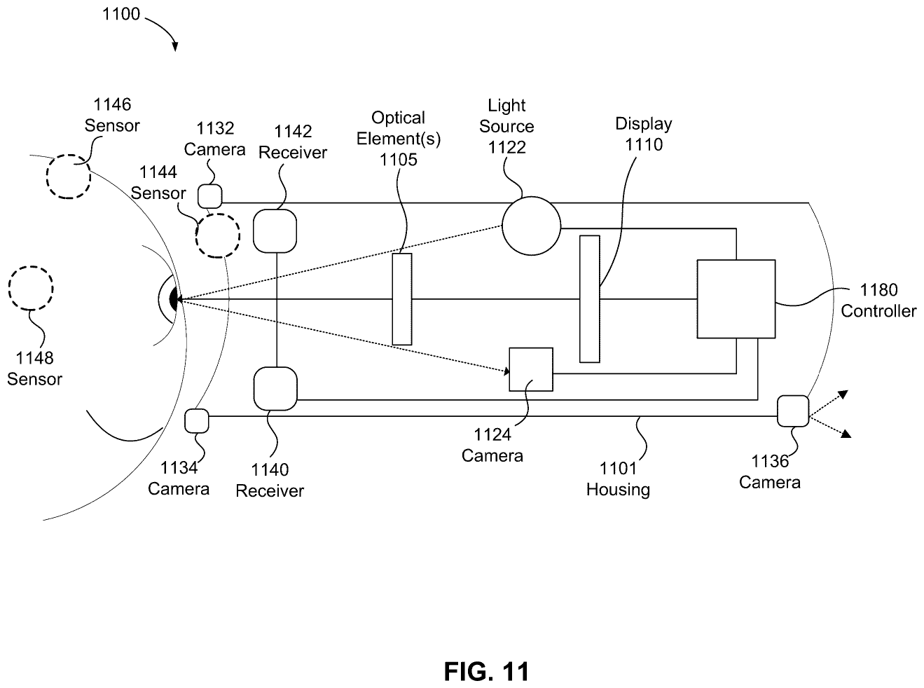

FIG. 11 illustrates a block diagram of an exemplary head-mounted device 1100 in accordance with some implementations. The head-mounted device 1100 includes a housing 1101 (or enclosure) that houses various components of the head-mounted device 1100. The housing 1101 includes (or is coupled to) an eye pad (not shown) disposed at a proximal (to the user) end of the housing 1101. In various implementations, the eye pad is a plastic or rubber piece that comfortably and snugly keeps the head-mounted device 1100 in the proper position on the face of the user (e.g., surrounding the eye of the user).

The housing 1101 houses a display 1110 that displays an image, emitting light towards or onto the pupil of an eye of a user. In various implementations, the display 1110 emits the light through an eyepiece having one or more optical elements 1105 that refracts the light emitted by the display 1110, making the display appear to the user to be at a virtual distance farther than the actual distance from the eye to the display 1110. For example, optical element(s) 1105 may include one or more lenses, a waveguide, other diffraction optical elements (DOE), and the like. For the user to be able to focus on the display 1110, in various implementations, the virtual distance is at least greater than a minimum focal distance of the eye (e.g., 7 cm). Further, in order to provide a better user experience, in various implementations, the virtual distance is greater than 1 meter.

The housing 1101 also houses a tracking system including one or more light sources 1122, camera 1124, and a controller 1180. The one or more light sources 1122 emit light onto the eye of the user that reflects as a light pattern (e.g., a circle of glints) that can be detected by the camera 1124. Based on the light pattern, the controller 1180 can determine an eye tracking characteristic of the user. For example, the controller 1180 can determine a gaze direction or a blinking state (eyes open or eyes closed) of the user. As another example, the controller 1180 can determine a pupil center, a pupil size, or a point of regard associated with the pupil. Thus, in various implementations, the light is emitted by the one or more light sources 1122, reflects off the eye of the user, and is detected by the camera 1124. In various implementations, the light from the eye of the user is reflected off a hot mirror or passed through an eyepiece before reaching the camera 1124.

The display 1110 emits light in a first wavelength range and the one or more light sources 1122 emit light in a second wavelength range. Similarly, the camera 1124 detects light in the second wavelength range. In various implementations, the first wavelength range is a visible wavelength range (e.g., a wavelength range within the visible spectrum of approximately 400-700 nm) and the second wavelength range is a near-infrared wavelength range (e.g., a wavelength range within the near-infrared spectrum of approximately 700-1400 nm).

In various implementations, eye tracking (or, in particular, a determined gaze direction) is used to enable user interaction (e.g., the user selects an option on the display 1110 by looking at it), provide foveated rendering (e.g., present a higher resolution in an area of the display 1110 the user is looking at and a lower resolution elsewhere on the display 1110), or correct distortions (e.g., for images to be provided on the display 1110). In various implementations, the one or more light sources 1122 emit light towards the eye of the user, which reflects in the form of a plurality of glints.

In various implementations, the camera 1124 is a frame/shutter-based camera that, at a particular point in time or multiple points in time at a frame rate, generates an image of the eye of the user. Each image includes a matrix of pixel values corresponding to pixels of the image which correspond to locations of a matrix of light sensors of the camera. In implementations, each image is used to measure or track pupil dilation by measuring a change of the pixel intensities associated with one or both of a user's pupils.

In various implementations, the housing 1101 also houses a camera system for capturing images of the user and/or the external environment. As illustrated in FIG. 11, the camera system may include camera 1132, camera 1134, and camera 1136. In various implementations, the camera 1132, camera 1134, and camera 1136 are frame/shutter-based cameras that, at a particular point in time or multiple points in time at a frame rate, can generate an image of the face of the user or capture an external physical environment. For example, camera 1132 captures images of the user's face below the eyes, camera 1134 captures images of the user's face above the eyes, and camera 1136 captures the external environment of the user (e.g., environment 100 of FIG. 1). The images captured by camera 1132, camera 1134, and camera 1136 may include light intensity images (e.g., RGB) or depth image data (e.g., Time-of-Flight, infrared, etc.).

In various implementations, the housing 1101 also houses a neurological sensing system (e.g., EEG monitoring) for capturing neurological signals of the user. As illustrated in FIG. 11, the neurological sensing system may include receivers 1140, 1142, and sensors 1144, 1146, and 1148. In various implementations, sensor 1144, sensor 1146, and sensor 1148 are electrodes for capturing various neurological signals that are received by the receiver 1140 and/or receiver 1142 either via wireless or direct electrical connections (wired). The receiver 1140 and/or receiver 1142 sends the neurological signals from the sensors to the controller 1180. In various implementations, the controller 1180 can determine various neural processes that are mediated through the electrical signals captured by the sensors.

In various implementations, the sensors 1144, 1146, and 1148 may be located on the outer surface of the housing 1101 (e.g., sensor 1144, which would contact the skin of the user when worn), or the sensors may be positioned on a head strap or other mounting system for the head worn device 1100 (e.g., sensors 1146, 1148, such that when the device 1100 is worn the sensors would contact the head of the user). The illustration of FIG. 11 is for illustrative purposes and is not meant to be limiting regarding the number or sensors and/or receivers, and the various positioning of the neurological sensing system components (e.g., location of the electrodes). For example, the electrodes of the neurological sensing system may be located on a separately worn device or head strap that could send the various neurological signals to the device 1100.

It will be appreciated that the implementations described above are cited by way of example, and that the present invention is not limited to what has been particularly shown and described hereinabove. Rather, the scope includes both combinations and sub combinations of the various features described hereinabove, as well as variations and modifications thereof which would occur to persons skilled in the art upon reading the foregoing description and which are not disclosed in the prior art.

As described above, one aspect of the present technology is the gathering and use of physiological data to improve a user's experience of an electronic device with respect to interacting with electronic content. The present disclosure contemplates that in some instances, this gathered data may include personal information data that uniquely identifies a specific person or can be used to identify interests, traits, or tendencies of a specific person. Such personal information data can include physiological data, demographic data, location-based data, telephone numbers, email addresses, home addresses, device characteristics of personal devices, or any other personal information.

The present disclosure recognizes that the use of such personal information data, in the present technology, can be used to the benefit of users. For example, the personal information data can be used to improve interaction and control capabilities of an electronic device. Accordingly, use of such personal information data enables calculated control of the electronic device. Further, other uses for personal information data that benefit the user are also contemplated by the present disclosure.

The present disclosure further contemplates that the entities responsible for the collection, analysis, disclosure, transfer, storage, or other use of such personal information and/or physiological data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates implementations in which users selectively block the use of, or access to, personal information data. That is, the present disclosure contemplates that hardware or software elements can be provided to prevent or block access to such personal information data. For example, in the case of user-tailored content delivery services, the present technology can be configured to allow users to select to “opt in” or “opt out” of participation in the collection of personal information data during registration for services. In another example, users can select not to provide personal information data for targeted content delivery services. In yet another example, users can select to not provide personal information, but permit the transfer of anonymous information for the purpose of improving the functioning of the device.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences or settings based on non-personal information data or a bare minimum amount of personal information, such as the content being requested by the device associated with a user, other non-personal information available to the content delivery services, or publicly available information.

In some embodiments, data is stored using a public/private key system that only allows the owner of the data to decrypt the stored data. In some other implementations, the data may be stored anonymously (e.g., without identifying and/or personal information about the user, such as a legal name, username, time and location data, or the like). In this way, other users, hackers, or third parties cannot determine the identity of the user associated with the stored data. In some implementations, a user may access his or her stored data from a user device that is different than the one used to upload the stored data. In these instances, the user may be required to provide login credentials to access their stored data.

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various objects, these objects should not be limited by these terms. These terms are only used to distinguish one object from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “or” as used herein refers to and encompasses any and all possible combinations of one or more of the associated listed items. It will be further understood that the terms “comprises” or “comprising,” when used in this specification, specify the presence of stated features, integers, steps, operations, objects, or components, but do not preclude the presence or addition of one or more other features, integers, steps, operations, objects, components, or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modification may be implemented by those skilled in the art without departing from the scope and spirit of the invention.

What is claimed is:

1. A method comprising:
at a device comprising a processor:
obtaining physiological data associated with neurological signals during presentation of content to a user at the device, wherein the content comprises an interaction element;
predicting, based on the physiological data, that the user is thinking of performing a physical act with a portion of a body of the user, wherein performance of the physical act is associated with a particular type of interaction and wherein performance of the physical act is detectable based on sensor data; and
determining user interaction feedback corresponding to an interaction event associated with the interaction element based on the predicting that the user is thinking of performing the physical act.

2. The method of claim 1, wherein predicting that the user is thinking of performing the physical act is based on recognizing that a pattern exhibited in the neurological signals of the physiological data is indicative of the user thinking of performing the physical act.

3. The method of claim 1, further comprising, and in response to determining, based on the sensor data, that the physical act associated with the particular type of interaction is performed:
obtaining additional physiological data associated with neurological signals; and
updating a prediction model associated with the user performing the physical act associated with the particular type of interaction based on the additional physiological data.

4. The method of claim 1, further comprising, and in response to determining, based on the sensor data, that the physical act associated with the particular type of interaction is not performed while the user is thinking of performing the physical act:
obtaining additional physiological data associated with neurological signals; and
updating a prediction model associated with the user performing the physical act associated with the particular type of interaction based on the additional physiological data.

5. The method of claim 1, wherein predicting that the user is thinking of performing the physical act with the portion of the body of the user is based on identifying a neurological event with at least one of the neurological signals.

6. The method of claim 5, wherein identifying the neurological event with the at least one neurological signal comprises determining whether one or more components of the at least one neurological signal comprises a change in one or more attributes with respect to a threshold.

7. The method of claim 1, wherein the physiological data associated with neurological signals comprises electroencephalogram (EEG) data.

8. The method of claim 1, wherein the device further comprises at least three neurological sensors configured to obtain the physiological data associated with neurological signals corresponding to motor cortex signals.

9. The method of claim 8, wherein each sensor of the at least three neurological sensors are positioned at different regions of the device.

10. The method of claim 9, wherein the different regions of the device that each of the at least three neurological sensors are positioned comprises a support element of the device, on a user facing surface adjacent to a lens of the device, via an additional support element coupled to the device, or a combination thereof.

11. The method of claim 1, wherein the user interaction feedback corresponding to an interaction event associated with the interaction element is classified using a machine learning technique.

12. The method of claim 1, wherein the physiological data comprises pupillary data, and wherein predicting that the user is thinking of performing the physical act is further based on determining a pupillary response associated with the interaction element.

13. The method of claim 12, wherein the pupillary response is:
a direction of the pupillary response;
a velocity of the pupillary response; or pupillary fixations.

14. The method of claim 1, wherein predicting that the user is thinking of performing the physical act with the portion of the body of the user is based on a combination of neurological signals corresponding to a neurological response and pupillary data corresponding to a pupillary response.

15. The method of claim 1, further comprising, prior to presenting the user interaction feedback, filtering a prediction value indicative of a motor intention through a damped-spring control function to generate a smoothed feedback signal.

16. The method of claim 15, wherein the smoothed feedback signal is based on at least one of a visual, haptic, and an auditory output presented to the user.

17. The method of claim 1, wherein the user interaction feedback comprises rendering a virtual representation of a portion of the user, the representation being animated toward the physical act in proportion to a confidence that the user is thinking of performing the physical act.

18. The method of claim 17, wherein the virtual representation is displayed adjacent to an interaction element predicted to be an intended target.

19. The method of claim 17, wherein updating the prediction model includes detecting a change in a motor-cortex signal after presentation of the virtual representation and adjusting model parameters based on the detected change.

20. The method of claim 1, further comprising:
modifying content in response to determining user interaction feedback corresponding to an interaction event associated with the interaction element based on the predicting that the user is thinking of performing the physical act.

21. The method of claim 1, further comprising:
obtaining additional physiological data associated with body movements; and
updating a prediction model associated with predicting that the user is thinking of performing the physical act associated with the particular type of interaction based on the additional physiological data.

22. A device comprising:
a non-transitory computer-readable storage medium; and
one or more processors coupled to the non-transitory computer-readable storage medium, wherein the non-transitory computer-readable storage medium comprises program instructions that, when executed on the one or more processors, cause the one or more processors to perform operations comprising:
obtaining physiological data associated with neurological signals during presentation of content to a user at the device, wherein the content comprises an interaction element;
predicting, based on the physiological data, that the user is thinking of performing a physical act with a portion of a body of the user, wherein performance of the physical act is associated with a particular type of interaction and wherein performance of the physical act is detectable based on sensor data; and
determining user interaction feedback corresponding to an interaction event associated with the interaction element based on the predicting that the user is thinking of performing the physical act.

23. A non-transitory computer-readable storage medium, storing program instructions executable by one or more processors on a device to perform operations comprising:
obtaining physiological data associated with neurological signals during presentation of content to a user at the device, wherein the content comprises an interaction element;
predicting, based on the physiological data, that the user is thinking of performing a physical act with a portion of a body of the user, wherein performance of the physical act is associated with a particular type of interaction and wherein performance of the physical act is detectable based on sensor data; and
determining user interaction feedback corresponding to an interaction event associated with the interaction element based on the predicting that the user is thinking of performing the physical act.
